(19) United States
(12) Patent Application Publication
(10) Pub. No.: US 2008/0027974 A1
(43) Pub. Date: Jan. 31, 2008

(54) Intensity modulated radiation therapy filtration apparatus, system and method

(76) Inventor: Donald W. Collins, Tempe, AZ (US)

Correspondence Address: TOD R NISSLE, PO BOX 55630, PHOENIX, AZ, 85078, US

(21) Appl. No.: 11/823,562

(22) Filed: Jun. 28, 2007

Related U.S. Application Data
(60) Provisional application No. 60/820,210, filed on Jul. 24, 2006.

(51) Int. Cl. G06F 9/00; A61N 5/10

(52) U.S. Cl. 707/102; 378/65

(57) ABSTRACT

Digital imaging and communications in a Medicine Radiation Therapy (DICOM RT) Cancer Patient Treatment Plan are converted into (i) multiple Treatment Field 2D Matrices; (ii) multiple IMRT Filter (Compensator) 3D Matrices; and (iii) 3D Computer Numerical Code (CNC) Files. From i, ii, and iii there is provided a set of (a) Intensity Modulated Radiation Therapy (IMRT) Filters; (b) IMRT Radiation Blocking Filters; and/or (c) IMRT Radiation Wound Protective Blocking Filters. The IMRT Radiation Blocking Filter can be used for each Cancer Patient's Treatment Field angle. The IMRT Radiation Wound Protective Blocking Filter can be used for each Cancer Patient's Surgical Incision. The foregoing filters can be used by a Radiation Oncology Hospital or Clinic to modulate a radiation dose or protect the surgical incision for a Cancer Patient for a specific radiation accelerator treatment machine.

### TECHNICAL FIELD

The present invention relates generally to radiation therapy, and is more particularly related to apparatus, methods and systems to provide Intensity Modulated Radiation Therapy (IMRT) Compensator Filters; IMRT Filters to modulate a radiation beam, IMRT Radiation Blocking Filters to block radiation around a Patient's Planned Treatment Volume, and Radiation Wound Protective Filters to protect a surgical wound by blocking radiation over a Surgical Incision; where the foregoing filters can be used in radiation accelerator treatment machines (both MLC Accelerators and Non-MLC Accelerators) providing intensity modulated radiation therapy (IMRT) for Cancer Patients.

### BACKGROUND

Intensity modulated radiation therapy (IMRT) is a cancer-fighting technology that uses software and hardware to direct a precise form of external beam radiation so as to irradiate and kill a cancerous growth. The software and hardware are used to vary the shape and intensity of radiation delivered to different parts of the treatment area where the cancerous growth is located.

IMRT links computed tomography scans to treatment planning software that allows the cancerous area to be visualized in three dimensions. In IMRT, a physician designates specific doses of radiation (constraints) that the cancerous growth and normal surrounding tissues should receive. A physics team then uses a computer program to develop an individualized plan to meet the constraints. This process is termed “inverse treatment planning.” IMRT uses a medical linear accelerator that delivers x-ray beams. A dynamic multi-leaf collimator (DMLC) is used in IMRT. The DMLC is a computer-controlled device that uses a plurality of movable “leaves” to conform the radiation beam to the shape of the cancerous area from any angle, while protecting normal adjacent tissue as much as possible. A DMLC allows the dose of radiation to vary within a single beam, thereby delivering higher radiation in some areas and lower radiation in others. The ability to vary the radiation dose with a DMLC is accomplished by “sliding windows” of radiation beams across the target cancerous area.

Though IMRT is a useful cancer-fighting technology, it is fraught with opportunities for human error. It would be an advance in the art to solve the problems of human error in IMRT that are heretofore unmet.

### BRIEF DESCRIPTION OF THE DRAWINGS

FIG. 1a is an exemplary implementation of a process flow via a Secure “Client” and “MSB” SFTP Servers;

FIG. 1b is an exemplary implementation of an intensity modulated radiation therapy (IMRT) Filter Label;

FIGS. 2a-2b depict a Compensator Negative Oncology Styrofoam or Polyurethane Negative Mold illustrated, respectively, in isometric and plan views;

FIGS. 3a-3b depict, respectively, isometric and plan views of an implementation of an Intensity Modulated Radiation Blocking Filter Negative Mold to be used in a radiation accelerator treatment machine;

FIGS. 4a-4b depict, respectively, isometric and plan views of an implementation of an Intensity Modulated Radiation Wound Protective Filter Negative Mold to be used in a radiation accelerator treatment machine;

FIGS. 5a-5b depict, respectively, isometric and plan views of an implementation of an IMRT Filter (Compensator) milled from solid Brass 360 to be used in a radiation accelerator treatment machine;

FIGS. 6a-6b depict, respectively, isometric and plan views of an implementation of an IMRT Filter (Compensator) milled from solid Aluminum 6061T6 to be used in a radiation accelerator treatment machine;

FIGS. 7a-7b depict, respectively, isometric and plan views of an implementation of an IMRT Radiation Blocking Solid Filter to be used in a radiation accelerator treatment machine;

FIGS. 8a-8b depict, respectively, isometric and plan views of an implementation of an IMRT Radiation Wound Protection Solid Filter to be used in a radiation accelerator treatment machine;

FIG. 9 depicts, in a block level diagram, three distinct implementations of process flows among and between (i) Client; (ii) IMRT Filter Supplier; and (iii) MSB;

FIG. 10 depicts a side view of a Block Tray with an IMRT Filter Mold and a Top Sealing Plate;

FIG. 11 depicts a Plan view of a Polycarbonate BLOCK Tray with Mounting Holes & Dimensions;

FIG. 12 depicts a Plan view of a Polycarbonate Top Sealing Plate with Holes & Dimensions;

FIGS. 13-17 depict respective side views of a Block Tray with an IMRT Filter Mold and the Top Sealing Plate used for calculating the Linear Attenuation Coefficient of the attenuating material;

FIG. 18 depicts, in a table of input fields, an exemplary user interface display screen for an IMRT Filter Compensator Service Registration;

FIG. 19 depicts, in a table of input fields for shipping/invoice/email address, contact names, and Tel/fax, an exemplary user interface display screen for an IMRT Filter Compensator Service Registration intended for use by radiation oncology units at hospitals or clinics;

FIG. 20 depicts, in a table of input fields for a Header File containing Information for a Patient Treatment Plan, an exemplary user interface display screen;

FIG. 21a depicts, in a table of input fields for a “Client” Order Tab, an exemplary user interface display screen;

FIG. 21b depicts, in a table of input fields for a Conversion Tab, an exemplary user interface display screen; and

FIG. 22 depicts, in a table of input fields for an “MSB” Manufacturing Tab, an exemplary user interface display screen.

### SUMMARY

Apparatus, methods and systems disclosed herein reduce the amount of human intervention when providing an Intensity Modulated Radiation Filter (IMRT Filter) for a specific radiation accelerator treatment machine for a Cancer Patient by electronically converting a Digital Imaging and Communications in Medicine Radiation Therapy (DICOM RT) Cancer Patient Treatment Plan into multiple Treatment Field 2D Matrices and into multiple IMRT Filters and/or IMRT Radiation Blocking Filters used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and for a specific Linear Attenuation Coefficient; into 3 Dimensional Matrices and into 3 Dimensional Computer Numerical Code (CNC) Files. This conversion process is automatically accomplished using an Automated Programmed Interface (API) via the Internet and a secure SFTP Server.

Implementations reduce the amount of human intervention when providing an Intensity Modulated Radiation Filter (IMRT) Filter; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and for a specific Linear Attenuation Coefficient by electronically converting a 3 Dimensional Compensator Files provided by the Radiation Oncology Hospital or Clinic that have Treatment Planning Systems that can produce Compensator Export Files into multiple IMRT Filter (Compensators) 3 Dimensional Computer Numerical Code (CNC) Files. This conversion process is automatically accomplished using an Automated Programmed Interface (API) via the Internet and a secure SFTP Server.

Implementations further provide methods for the conversion of a Digital Imaging and Communications in Medicine Radiation Therapy (DICOM RT) Cancer Patient Treatment Plan into multiple Treatment Field 2D Matrices and into multiple IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and for a specific Linear Attenuation Coefficient (Compensator) 3 Dimensional Matrices and into multiple 3 Dimensional Computer Numerical Code (CNC) Files, wherein the method comprises receiving a Digital Imaging and Communications in Medicine Radiation Therapy (DICOM RT) Cancer Patient Treatment Plan. This conversion process is automatically accomplished using an Automated Programmed Interface (API) via the Internet and a secure “Client” SFTP Server.

Implementations still further provide methods for defining the tooling instructions for the selected Intensity Modulated Radiation Filter (IMRT Filter) blank, and automatically programming the XYZ coordinates for milling the set of IMRT Filter Compensators on a Computer Numerical Control (CNC) machine based upon the defined tooling instructions. This conversion process is automatically accomplished using an Automated Programmed Interface (API) via the Internet and a secure “MSB” SFTP Server.

Implementations also provide methods for the conversion of a 3 Dimensional Compensator Cancer Patient Treatment Field provided by the Radiation Oncology Hospital or Clinic that has Treatment Planning Systems that can produce Compensator Export Files into multiple IMRT Filter (Compensator) and/or IMRT Radiation Blocking Filter 3 Dimensional Computer Numerical Code (CNC) Files and into multiple 3 Dimensional Computer Numerical Code (CNC) files for a specific radiation accelerator treatment machine, and a specific attenuating material, for a specific Tray location. This conversion process is automatically accomplished using an Automated Programmed Interface (API) via the Internet and a secure “Client” SFTP Server.

Implementations still further provide methods for defining tooling instructions for the selected Intensity Modulated Radiation Filter (IMRT Filter) blank, and automatically programming the XYZ coordinates for milling the IMRT Filter on a CNC machine based upon the defined tooling instructions. This conversion process is automatically accomplished using an Automated Programmed Interface (API) via the Internet and a secure “MSB” SFTP Server.

An advantage of electronically converting the received DICOM RT Cancer Patient Treatment Plan Data into the “Converter Database” is that this is done without human intervention; it is done via the secure “Client” SFTP Server using an Automated Programming Interface (API). Each of the following is automatically selected in the Database: Intensity Modulated Radiation Filter (IMRT Filter) blank; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and for a specific Linear Attenuation Coefficient.

Each type of radiation accelerator treatment machine may have a maximum IMRT Filter size requirement determined by the Source to Tray Z-Distances for the upper wedge tray, block tray and lower wedge tray and the X1-X2 Y1-Y2 Tray (XY) and Thickness Dimensions for each tray. These Z-Distances selected for a specific “Client” and specific radiation accelerator treatment machine are stored in the “Client” Database during the “Commissioning Process”.

The compatibility of the selected IMRT Filter blank is based upon meeting the IMRT Filter size requirement of the intended radiation accelerator treatment machine using the selected attenuating material and calculated linear attenuation coefficient (LAC) and the Source to Tray Z-Distance for the selected Tray Mounting Position and the Source to Patient Isocenter Distance. These values are stored in the “Converter Database” during the “Commissioning Process”. The advantage of using an Automated Programming Interface (API) to automatically select the IMRT Filter size requirement is therefore to eliminate any possible human error.

The advantage of building a secure “MSB” SFTP Server connected to the “Converter Database” using an Automated Programming Interface (API) to provide a secure Cancer Patient IMRT Filter Compensator Service by an “MSB Evaluation Process”, which includes; establishing a “Manufacturing Service Bureau” ID, “MSB” Address, “MSB” contact information, “MSB” Password, “MSB” CNC Machine IDs, the Number of CNC Machines, the “Rough Cut” Tool, the “Finish Cut” Tool, the “Location Hole” Tool, the Engraving Tool, and the “Inspection Probe” Tool is to allow the removal of material to form the IMRT Filter in the tooling database by electronically selecting the five separate tools to apply the five separate machining tasks in the CNC milling Instruction of an IMRT Filter and by providing 6-sigma quality control to meet acceptable tolerances.

The advantage of building a secure “MSB” SFTP Server “Converter Database” using an Automated Programming Interface (API) is when the next “MSB” CNC Machine becomes available, the “MSB” Manufacturing Technician uses a Barcode Reader to scan a label of the next IMRT Filter Blank in the “MSB” FIFO Production Queue to retrieve the CNC Milling Code Milling Instructions from the “Converter Database” to mill the IMRT Filter Compensator for a specific “Client”, for a specific accelerator, for a specific Attenuating Material, for a specific Tray Location, and for this Patient's specific Treatment Angle, via the secure “MSB” SFTP Server. This method eliminates any possibility of Human Error.

### DESCRIPTION

Implementations provide apparatus, methods and systems to provide Intensity Modulated Radiation Therapy using a set of Intensity Modulated Radiation Filters (IMRT Filters); IMRT Filters; and/or IMRT Radiation Blocking Filters. The foregoing filters can be used for each Cancer Patient's Treatment Field angle; and/or Radiation Wound Protective Blocking Filter used for each Cancer Patient's Surgical Incision, for a specific radiation accelerator treatment machine for a specific Radiation Oncology Hospital or Clinic. Within these implementations there are included all or a partial set of the following thirty-six (36) manipulative steps:

First; establishing an approved Radiation Oncology Hospital or Clinic (hereinafter referred to as “Client”); “Client” ID, “Client” Address, “Client” Contact Information, “Client” Accelerator ID, selection of an attenuating material for the “Client” (e.g.; Tungsten Metal Powder, Cerrobend, Aluminum 6061T6, Brass 360, etc.), Linear Attenuation Coefficient (LAC) calculated for that attenuating material and specific radiation accelerator treatment machine, the Tray selected for mounting the IMRT Filter (Upper Wedge Tray Distance from Source, Wedge Tray Thickness, Block Tray Distance from Source, Block Tray Thickness, Lower Wedge Tray Distance from Source, Lower Wedge Tray Thickness), the Isocenter Distance from Source, Isocenter Calibration Markings (X1 X2 and Y1 Y2), on the selected Tray, the IMRT Filter mounting position (source side of selected Tray or patient side of selected Tray) and assigning a “Client” Password in the “Converter Database” for access to the Secure “Client” SFTP Server, and

Second; establishing an approved Manufacturing Service Bureau (hereinafter referred to as “MSB”); “MSB” ID, “MSB” Address, “MSB” Contact Information, “MSB” CNC Machine IDs, “MSB” Number of CNC Machines, “MSB” Rough Cut Tool Set, “MSB” Finish Cut Tool Set, “MSB” Location Hole Tool Set, “MSB” Engraving Tool Set, “MSB” Tolerance Calibration Tool Set and assigning a “MSB” Password in the “Converter Database” for access to the secure “MSB” SFTP Server, and

Third; receiving (importing) from a “Client” via the Internet using a secure “Client” SFTP Server, the DICOM RT Cancer Patient Treatment Plan File, which includes the multiple Optimal Fluence Field Matrix data for the multiple Treatment Field angles, and

Fourth; the conversion of the DICOM RT Cancer Patient Treatment Plan File into a DICOM RT Cancer Patient Treatment Plan File with IMRT Filters (Compensators—with Thickness Matrices and Transmission Matrices in the DICOM format) for return (export) to the specific “Client”, via the Internet using a secure “Client” SFTP Server for “Dose” Calculations, and

Fifth; the conversion of the Optimal Fluence Field data for each Cancer Patient's Treatment Field angle into a 3 Dimensional Computer Numerical Code (CNC “G-Code”), Machine Control File, to be used to automatically control the XYZ Tool movement in the milling of the Intensity Modulated Radiation Filter (IMRT Filter); IMRT Filter and/or IMRT Radiation Blocking Filter used for each Cancer Patient's Treatment Field angle; and/or Radiation Wound Protective Blocking Filter used for each Cancer Patient's Surgical Incision, for a specific radiation accelerator treatment machine, for a specific Radiation Oncology Hospital or Clinic “Client”, and

Sixth; sending (exporting) the CNC Machine Control File (CNC “G-Code”) via the Internet using a secure “MSB” SFTP Server to the Manufacturing Service Bureau (“MSB”) for manufacturing the Cancer Patient's Set of Intensity Modulated Radiation Filters (IMRT Filters); IMRT Filter and/or IMRT Radiation Blocking Filter used for each Cancer Patient's Treatment Field angle; and/or Radiation Wound Protective Blocking Filter used for each Cancer Patient's Surgical Incision, for a specific radiation accelerator treatment machine, for a specific Radiation Oncology Hospital or Clinic “Client”, and

Seventh; receiving (importing) from a “Client”, via the Internet using a secure “Client” SFTP Server, 3 Dimensional Compensator Files, from “Clients” that have Treatment Planning Systems that can produce 3 Dimensional Compensator Files for export of a Cancer Patient's Treatment Plan multiple fields, and

Eight; the conversion of the 3 Dimensional Compensator files received (imported) from “Clients” into 3 Dimensional Computer Numerical Code (CNC) Machine Control Files for manufacturing the Cancer Patient's Set of Intensity Modulated Radiation Filters (IMRT Filters); IMRT Filter and/or IMRT Radiation Blocking Filter used for each Cancer Patient's Treatment Field angle, for a specific accelerator, for a specific Radiation Oncology Hospital or Clinic “Client”, and

Ninth; sending (exporting), via the Internet using a secure “MSB” SFTP Server, to the Manufacturing Service Bureau (“MSB”) the multiple 3 Dimensional Computer Numerical Code (CNC “G-Code”) Machine Control Files to be used to control the milling of the Intensity Modulated Radiation Filters (IMRT Filters); IMRT Filter and/or IMRT Radiation Blocking Filter used for each Cancer Patient's Treatment Field angle, for a specific accelerator, for a specific Radiation Oncology Hospital or Clinic “Client”, and

Tenth; sending (exporting) to the Radiation Oncology Hospital or Clinic (e.g.; “Client”) via the Internet using a secure “Client” SFTP Server, an Invoice for the Cancer Patient's Treatment Plan set of IMRT Filters and/or IMRT Radiation Blocking Filters and/or Radiation Wound Protective Blocking Filter, and

Eleventh; sending (exporting) to the Manufacturing Service Bureau, “MSB”, via the Internet using a secure “MSB” SFTP Server, a Purchase Order File (PO) for the “MSB” which includes the following xml files:
(i) IMRT Filter Type (IMRT Filters and/or IMRT Radiation Blocking Filters and/or Radiation Wound Protective Blocking Filter);
(ii) IMRT Filter Attenuating Material Type (e.g.; Polyurethane, Brass, Aluminum, etc.);
(iv) IMRT Filter Label File
(v) “Client” Shipping Label File; and
(vi) IMRT Filter CNC “G-Code” Files
(vii) IMRT Filter Quality Analysis Test Files.

Twelfth; sending (exporting) to the Manufacturing Service Bureau, “MSB”, via the Internet using a secure “MSB” SFTP Server, an Alpha/Numeric and Barcode Label File for the Cancer Patient's Treatment Plan set of IMRT Filters and/or IMRT Radiation Blocking Filters used for each Cancer Patient's Treatment Field angle; and/or Radiation Wound Protective Blocking Filters used for each Cancer Patient's Surgical Incision, including a two inch by eight inch IMRT Filter Alpha/Numeric and Barcode Label for each Cancer Patient's Treatment Angle and a Shipping/Confirmation Alpha/Numeric and Barcode Label for a specific accelerator, for a specific Radiation Oncology Hospital or Clinic “Client”, and

Thirteenth; sending (exporting) to the Manufacturing Service Bureau, “MSB”, via the Internet using a secure “MSB” SFTP Server, the multiple 3 Dimensional Computer Numerical Code (CNC “G-Code”) Machine Control Files to be used to control the milling of the IMRT Filters and/or IMRT Radiation Blocking Filters used for each Cancer Patient's Treatment Field angles; and/or Radiation Wound Protective Blocking Filters used for each Cancer Patient's Surgical Incision, for a specific accelerator and for a specific Radiation Oncology Hospital or Clinic “Client”, and

Fourteenth; sending (exporting) to the Manufacturing Service Bureau, “MSB”, via the Internet using a secure “MSB” SFTP Server, the multiple 3 Dimensional Computer Numerical Code (CNC “G-Code”) Machine Quality Analysis (QA) Nine-Point Probe Test Files to be used for quality control tolerance inspection of each of the IMRT Filters and/or IMRT Radiation Blocking Filters used for each Cancer Patient's Treatment Field angle; and/or Radiation Wound Protective Blocking Filters used for each Cancer Patient's Surgical Incision, for a specific accelerator, and for a specific Radiation Oncology Hospital or Clinic “Client”, and

Fifteenth; the “MSB” prints the IMRT Filter Label File and attaches the labels to the IMRT Filter Blanks, The IMRT Filter Blanks can be selected from the following materials, Oncology Styrofoam Blocks, or Polyurethane Blocks, or Aluminum 6061T6 and or Brass 360 Blocks, and

Sixteenth; the “MSB” prints the “Client” Shipping/Confirmation Label File and attaches the Shipping Label/s to the Shipping Carton/s, and

Seventeenth; the “MSB” places the IMRT Filter Blanks with Labels attached in a First-In-First-Out (FIFO) Manufacturing Production Queue, and

Eighteenth; the “MSB” places the IMRT Filter Shipping Carton/s with Labels attached in a First-in-First-Out (FIFO) Shipping Queue, and

Nineteenth; when a CNC Machine becomes available, the “MSB” places the next IMRT Filter Blank from the Production FIFO Queue into the CNC Machine Fixture for milling. The Technician uses a Barcode Reader to scan the label attached to the IMRT Filter Blank to automatically retrieve the CNC Milling Code from the “Converter Database”, via the secure “MSB” SFTP Server, to mill the IMRT Filter and/or IMRT Radiation Blocking Filter used for each Cancer Patient's Treatment Field angle; and/or Radiation Wound Protective Blocking Filter used for each Cancer Patient's Surgical Incision, for a specific radiation accelerator machine, in the Negative for Compensator Molds (made from Polyurethane or Oncology Styrofoam) and in the Positive for Solid Compensators (made from Brass 360 or Aluminum 6061T6), and

Twentieth; once the four milling process steps (Rough Cut, Finish Cut, Location Hole Cut and Engraving) are complete, the “MSB” does a Quality Analysis (QA) Test of the IMRT Filter using the 3 Dimensional Computer Numerical Code (CNC “G-Code”) Machine Quality Analysis (QA) Nine-Point Test File to verify the accuracy (acceptable tolerance) of the IMRT Filter Compensator prior to removal from the CNC machine, and

Twenty-first; if the acceptable tolerances are met, the “MSB” cleans the IMRT Filter and/or IMRT Radiation Blocking Filter used for each Cancer Patient's Treatment Field angle; and/or Radiation Wound Protective Blocking Filter used for each Cancer Patient's Surgical Incision, for a specific radiation accelerator machine, for a specific “Client”, for shipping and places it at the end of the “MSB” FIFO Shipping Queue, and

Twenty-second; the “MSB” exports the Quality Analysis (QA) Nine-Point Probe Test Results Report back to the Converter for storage in the “Converter Database”, via the Internet using a secure “MSB” SFTP Server, and

Twenty-third; if the acceptable tolerances are NOT met, the “MSB” destroys the IMRT Filter, does a CNC Machine Inspection to determine the error, makes the necessary correction/s, prints a new IMRT Filter Label, attaches the label to an IMRT Filter Blank, places the IMRT Filter Blank in front of the “MSB” FIFO Production Queue for the next available CNC Machine, and

Twenty-fourth; the “MSB” Shipping Clerk uses the Barcode Reader on the next IMRT Filter and/or IMRT Radiation Blocking Filter used for each Cancer Patient's Treatment Field angle; and/or Radiation Wound Protective Blocking Filter used for each Cancer Patient's Surgical Incision, in the “MSB” FIFO Shipping Queue and packages the set of IMRT Filters with the matching labels for that Cancer Patient and for that “Client” into the Matching Barcode Shipping Carton/s, and

Twenty-fifth; the “MSB” ships the Carton/s of the completed set of IMRT Filters for the Cancer Patient to the “Client” and sends (exports) the Shipping/Confirmation and Tracking Number to the “Client” and the Converter, and

Twenty-sixth; the “Client” receives the Carton/s of Negative Mold IMRT Filters. The Radiotherapist opens the Carton/s and uses a Barcode Reader on each IMRT Filter, which automatically accesses the “Converter Database” via the Internet using a secure “Client” SFTP Server and retrieves the instruction information on how to fill the Negative Mold for the IMRT Filter and/or IMRT Radiation Blocking Filter used for each Cancer Patient's Treatment Field angle; and/or Radiation Wound Protective Blocking Filter used for each Cancer Patient's Surgical Incision, for a specific accelerator, with the exact attenuating material, Tungsten Metal Powdered Metal or molten Cerrobend, and how to mount the IMRT Filter on the correct Tray, and

Twenty-seventh; the “Client” receives the Carton of Solid IMRT Filters. The Radiation Therapist opens the Carton/s and uses a Barcode Reader on each IMRT Filter, which automatically accesses the “Converter Database” via the Internet using a secure “Client” SFTP Server and retrieves the instruction information to fill the Solid IMRT Filter for the IMRT Filter and/or IMRT Radiation Blocking Filter used for each Cancer Patient's Treatment Field angle; and/or Radiation Wound Protective Blocking Filter used for each Cancer Patient's Surgical Incision, for a specific accelerator, with the exact attenuating material, Brass 360 or Aluminum 6016T6, and how to mount the IMRT Filter on the correct Tray, and

Twenty-eighth; the “Client” inserts the Compensator Filter mounted on the tray using the instructions from the “Converter Database” into the correct accessory holder of the radiation accelerator treatment machine for that Tray and does a Dosimetric Film Test, and

Twenty-ninth; if the Dosimetric Film Test is approved by the Radiation Oncologist, the filled Negative Mold IMRT Filter or the Solid IMRT Filter is now ready to be used for the Cancer Patient's treatment cycle. The set of Patient IMRT Filters are stored in the “Client” wall mounted IMRT Filter Storage Rack with the IMRT Filter Barcode Labels visible, and

Thirtieth; the “Client” send (exports) the Dosimetric Film Test acceptance results to the “Converter Database”, via the secure “Client” SFTP Server, and

Thirty-first; The Converter archives the Dosimetric Film Test data for each Cancer Patient's IMRT Filter and/or IMRT Radiation Blocking Filter used for each Cancer Patient's Treatment Field angle; and/or Radiation Wound Protective Blocking Filter used for each Cancer Patient's Surgical Incision, for a specific accelerator, for a specific Radiation Oncology Hospital or Clinic “Client”, with the exact attenuating material, within the “Converter Database”.

Thirty-second; the “Client” prepares the Patient for the normal thirty days of treatment, five days a week over six weeks by attaching a Wrist Band with Bar-coded information that matches the “Client” Treatment Planning Database and the Converter Database. This Wrist Band will be worn by the Patient for the thirty days of treatment, and

Thirty-third; the Patient arrives at the “Client” location for his/her radiation treatment. The Radiotherapist assists the Patient to lie down on the treatment table. Selects the first IMRT Filter by Patient Name from the IMRT Filter Storage Rack and inserts the IMRT Filter into the radiation accelerator treatment machine Tray Slot. The Barcode Reader is first used to determine if this IMRT Filter matches the Patient's wrist band. If automatically approved, the second step is to verify that the IMRT Filter is placed in the correct Tray Slot. If automatically approved, the third step is to automatically rotate the radiation accelerator treatment machine to the correct angle for this Patient's Treatment Field. The Radiotherapist leaves the treatment room and the radiation dose is given automatically. The “Client” Database is automatically updated for this IMRT Filter for this specific treatment angle with the treatment date. This IMRT Filter cannot be used twice on same day eliminating any possible Human Error, and

Thirty-fourth; the Radiotherapist enters the treatment room and selects the next IMRT Filter by Patient Name from the IMRT Filter Storage Rack and inserts the IMRT Filter into the radiation accelerator treatment machine Tray Slot. The Barcode Reader is again first used to determine if this IMRT Filter matches the Patient's wrist band. If automatically approved, the second step is to verify that the IMRT Filter is placed in the correct Tray Accessory Slot. If automatically approved, the third step is to automatically rotate the radiation accelerator treatment machine to the correct angle for this Patient's Treatment Field. The Radiotherapist leaves the treatment room and the radiation dose is given automatically. The “Client” Database is automatically updated for this IMRT Filter for this specific treatment angle with the treatment date. This IMRT Filter cannot be used twice on same day eliminating any possible Human Error, and

Thirty-fifth; steps Thirty-third and Thirty-fourth are repeated until all the IMRT Filters for this Patient have been accessed on this Treatment Date. The Radiotherapist cannot treat another patient until the “Client” Database automatically approves the completion of this Patient's Treatment for this Date. This also eliminates any possible Human Error, and

Thirty-sixth; steps Thirty-third and Thirty-fourth and Thirty-fifth are repeated for this Patient five days a week for the total of six weeks, thirty treatment plan days. The Radiotherapist cannot treat this patient for a thirty first day because the “Client” Database automatically locks this Patient's Treatment Plan Files when the last Treatment Date is finished. This also eliminates any possible Human Error.

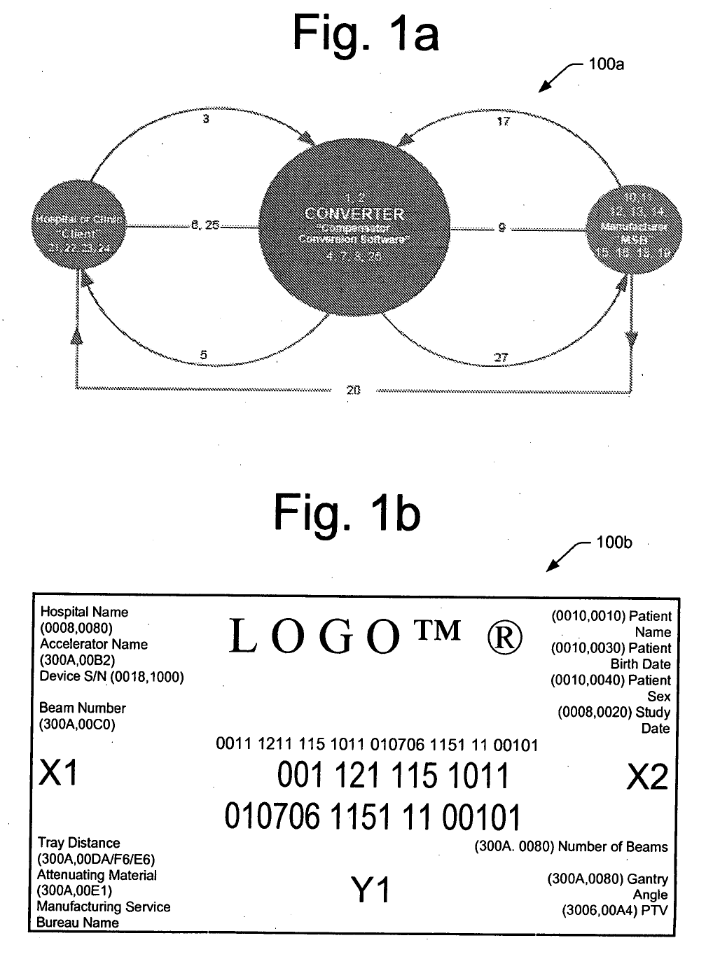

The following paragraphs define, for an implementation of an inventive IMRT process flow an example of which is seen in FIG. 1a, the Device Names, the Device Raw Materials, the Statement of Intended Use, the Digital Imaging and Communications in Medicine Radiation Therapy (DICOM RT), the Converter's implementation of DICOM RT, the Device Descriptions, and the Technological Characteristics.

Device Names

Intensity Modulating Radiation Therapy Compensators including:

IMRT Filter

IMRT Radiation Blocking Filter

Radiation Wound Protective Blocking Filter

Device Raw Materials

Intensity Modulating Radiation Therapy Compensators can be manufactured from the following radiation attenuating materials:

Polyurethane Blocks

Oncology Styrofoam Blocks

Brass 360 Blocks

Aluminum 6061T6 Blocks

Statement of Intended Use

Intensity Modulating Radiation Therapy Compensators (IMRT Filters); IMRT Filter and/or IMRT Radiation Blocking Filter used for each Cancer Patient's Treatment Field angle; are indicated to be used for external beam radiation therapy for Cancer Patients to modulate the intensity and shape of a radiation beam to compensate for missing tissue, for tissue heterogeneities, or to protect underlying tissue by intensity modulation, i.e. Intensity Modulated Radiation Therapy (IMRT); and/or Radiation Wound Protective Blocking Filter used for each Cancer Patient's Surgical Incision, used to protect the Surgical Incision of those Cancer Patients requiring both surgery and external beam radiation therapy.

DICOM RT

DICOM RT (Radiation Therapy) is used to handle the transfer of data unique to the Radiation Oncology arena. The Converter supports the following four objects that represent the current recognized DICOM RT standard:
RT Structure set—Information related to anatomy such as Isocenters and markers.
RT Plan—Geometric and dosimetric data such as external beam components.
RT Dose—Dose data such as reference points and isodose curves.
RT Treatment Record—Historical record of all treatment data such that the process may be re-created at any given point in time.

The Converter's implementation of DICOM RT includes:

The Converter's DICOM RT is capable of Send, Receive, Query/Retrieve and Print both as a user and a provider.
DICOM Send (Service Class User)—allows supported* RT objects to be sent to another DICOM RT device.
DICOM Receive (Service Class Provider)—allows supported* RT objects to be received by another DICOM RT device.
DICOM Query/Retrieve—allows the user to interrogate another DICOM device and retrieve supported* data.
DICOM Print—allows RT objects to be sent to a DICOM compatible printer.

Device Descriptions

An “IMRT Filter (Compensator Mold)” consists of a precision milled reverse Polyurethane Mold or Oncology Styrofoam Mold with Radiation Accelerator X1 X2 and Y1 Y2 orientation and Isocenter markings and CNC Machine Serial ID# engraved on the Source Side of each IMRT Filter. Each IMRT Filter Mold is filled with Tungsten Metal Powder or Molten Cerrobend, each labeled with a “Client” Name, “Client” Accelerator Name, “Client” Accelerator Device Serial Number, “Client” Radiation Beam Number, “Client” Attenuating Material, “Client” Linear Attenuation Coefficient (LAC), Cancer Patient Name, Cancer Patient Birth Date, Cancer Patient Sex, Cancer Patient Study Date, Total Number of Beams, Gantry Angle for this IMRT Filter, and Planned Target Volume (PTV). All the above label alpha/numeric data will be included in the barcode centrally located within the label. The IMRT Filter Compensator device is placed in the linear accelerator beam path between the radiation source and the Cancer Patient by mounting it to a specific accelerator tray.

The “IMRT Filter (Compensator Solid)” can also be precision milled from a solid block of Aluminum (6061T6) or a solid block of Brass 360 with Radiation Accelerator X1 X2 and Y1 Y2 orientation and Isocenter markings and CNC Machine Serial ID# engraved on the Source Side of each Solid IMRT Filter. Each filter is labeled with a “Client” Name, “Client” Accelerator Name, “Client” Accelerator Device Serial Number, “Client” Radiation Beam Number, “Client” Attenuating Material, “Client” Linear Attenuation Coefficient (LAC), Cancer Patient Name, Cancer Patient Birth Date, Cancer Patient Sex, Cancer Patient Study Date, Total Number of Beams, Gantry Angle for this IMRT Filter, and Planned Target Volume. All the above label alpha/numeric data will be included in the barcode centrally located within the label. The IMRT Filter Compensator device is placed in the linear accelerator beam path between the radiation source and the Cancer Patient by mounting it to a specific accelerator tray.

An “IMRT Radiation Blocking Filter (Compensator Mold)” consists of a precision milled reverse Polyurethane Mold or Oncology Styrofoam Mold with Radiation Accelerator X1 X2 and Y1 Y2 orientation and Isocenter markings and CNC Machine Serial ID# engraved on the Source Side of each IMRT Filter. Each IMRT Filter Mold is filled with Tungsten Metal Powder or Molten Cerrobend, each labeled with a “Client” Name, “Client” Accelerator Name, “Client” Accelerator Device Serial Number, “Client” Radiation Beam Number, “Client” Attenuating Material, “Client” Linear Attenuation Coefficient (LAC), Cancer Patient Name, Cancer Patient Birth Date, Cancer Patient Sex, Cancer Patient Study Date, Total Number of Beams, Gantry Angle for this IMRT Filter, and Planned Target Volume. All the above label alpha/numeric data will be included in the barcode centrally located within the label. The IMRT Filter Compensator device is placed in the linear accelerator beam path between the radiation source and the Cancer Patient by mounting it to a specific accelerator tray.

The “IMRT Radiation Blocking Filter (Compensator Solid)” can also be precision milled from a solid block of Aluminum (6061T6) or a solid block of Brass 360 with Radiation Accelerator X1 X2 and Y1 Y2 orientation and Isocenter markings and CNC Machine Serial ID# engraved on the Source Side of each Solid IMRT Filter. Each filter is labeled with a “Client” Name, “Client” Accelerator Name, “Client” Accelerator Device Serial Number, “Client” Radiation Beam Number, “Client” Attenuating Material, “Client” Linear Attenuation Coefficient (LAC), Cancer Patient Name, Cancer Patient Birth Date, Cancer Patient Sex, Cancer Patient Study Date, Total Number of Beams, Gantry Angle for this IMRT Filter and Planned Target Volume. All the above label alpha/numeric data will be included in the barcode centrally located within the label. The IMRT Filter Compensator device is placed in the linear accelerator beam path between the radiation source and the Cancer Patient by mounting it to a specific accelerator tray.

An “Radiation Wound Protective Blocking Filter (Compensator Mold)” used for each Cancer Patient's Surgical Incision, consists of a precision milled reverse Polyurethane Mold or Oncology Styrofoam Mold with Radiation Accelerator X1 X2 and Y1 Y2 orientation and Isocenter markings and CNC Machine Serial ID# engraved on the Source Side of each IMRT Filter. Each IMRT Filter Mold is filled with Tungsten Metal Powder or Molten Cerrobend, each labeled with a “Client” Name, “Client” Accelerator Name, “Client” Accelerator Device Serial Number, “Client” Radiation Beam Number, “Client” Attenuating Material, “Client” Linear Attenuation Coefficient (LAC), Cancer Patient Name, Cancer Patient Birth Date, Cancer Patient Sex, Cancer Patient Study Date, Total Number of Beams, Gantry Angle for this IMRT Filter, and Planned Target Volume. All the above label alpha/numeric data will be included in the barcode centrally located within the label. The IMRT Filter Compensator device is placed in the linear accelerator beam path between the radiation source and the Cancer Patient by mounting it to a specific accelerator tray.

The “Radiation Wound Protective Blocking Filter (Compensator Solid)” used for each Cancer Patient's Surgical Incision, can also be precision milled from a solid block of Aluminum (6061T6) or a solid block of Brass 360 with Radiation Accelerator X1 X2 and Y1 Y2 orientation and Isocenter markings and CNC Machine Serial ID# engraved on the Source Side of each Solid IMRT Filter. Each filter is labeled with a “Client” Name, “Client” Accelerator Name, “Client” Accelerator Device Serial Number, “Client” Radiation Beam Number, “Client” Attenuating Material, “Client” Linear Attenuation Coefficient (LAC), Cancer Patient Name, Cancer Patient Birth Date, Cancer Patient Sex, Cancer Patient Study Date, Total Number of Beams, and Gantry Angle for this IMRT Filter, and Planned Target Volume. All the above label alpha/numeric data will be included in the barcode centrally located within the label. The IMRT Filter Compensator device is placed in the linear accelerator beam path between the radiation source and the Cancer Patient by mounting it to a specific accelerator tray.

Technological Characteristics

There has been produced a DICOM RT Conversion Software used to convert DICOM RT Cancer Patient Treatment Plans into DICOM RT Cancer Patient Treatment Plan IMRT Filter Compensators and Optimal Fluence Plan Field Matrices for each Treatment Angle and automatically producing the 3 Dimensional CNC Code for the manufacture of Intensity Modulated Radiation Therapy (IMRT Filters); IMRT Filter and/or IMRT Radiation Blocking Filter used for each Cancer Patient's Treatment Field angle; and/or Radiation Wound Protective Blocking Filter used for each Cancer Patient's Surgical Incision, from a reverse (negative) cut Oncology Styrofoam or Polyurethane Mold Block to be filled with Tungsten Powdered Metal or Molten Cerrobend or from a positive cut solid Aluminum (6061T6) Block, or from a positive cut solid Brass 360 Block.

There has also been produced a Compensator Conversion Software for those “Clients” that have Vendor Treatment Planning Software that can export Compensator Files for each Treatment Angle and automatically produce the 3 Dimensional CNC Code for the manufacture of Intensity Modulated Radiation Therapy (IMRT Filters); IMRT Filter and/or IMRT Radiation Blocking Filter used for each Cancer Patient's Treatment Field angle; and/or Radiation Wound Protective Blocking Filter used for each Cancer Patient's Surgical Incision, from a reverse (negative) cut Oncology Styrofoam or Polyurethane Mold Block to be filled with Tungsten Powdered Metal or Molten Cerrobend or from a positive cut solid Aluminum (6061T6) Block, or from a positive cut solid Brass 360 Block.

Process Flow: FIG. 1a

Turning now to FIG. 1a, an exemplary implementation of an inventive IMRT process flow via a Secure “Client” and “MSB” SFTP Servers is depicted in Step 1 through Step 27, as follows:

Step-1 Establish a Radiation Oncology Hospital or Clinic, “Client” in the “Converter Database”.

Step-2 Establish a Manufacturing Service Bureau, “MSB” in the “Converter Database”.

Step-3 The “Client” exports the DICOM RT Cancer Patient Treatment Plan File or exports the 3 Dimensional Compensator Files to the “Filter Supplier”.

Step-4 The “Filter Supplier” imports the DICOM RT Cancer Patient Treatment File and converts it into multiple Treatment Field 2D Matrices and into multiple IMRT Filter (Compensator) 3 Dimensional Matrices, and into multiple 3 Dimensional Computer Numerical Code (CNC) Machine Control Files; or

The “Filter Supplier” imports the 3 Dimensional Compensator Files; from “Clients” that have Treatment Planning Systems that can produce 3 Dimensional Compensator Files for a Cancer Patient's Treatment Plan and converts them into multiple 3 Dimensional Computer Numerical Code (CNC) Machine Control Files;

Step-5 The “Filter Supplier” exports the IMRT Filter DICOM RT Compensator File which includes the Thickness Matrix and Transmission Matrix for each Treatment Field back to the “Client” for the Dose Calculation and approval;

Step-6 The “Client's” Radiologist and Medical Physicist determines that the Dose Calculations are correct and approves the IMRT Filter Compensator File for milling and informs the “Filter Supplier”;

Step-7 The “Filter Supplier” then converts the IMRT Filter DICOM RT Compensator File or “Client” Compensator File into 3 Dimensional CNC Milling Code;

Step-8 The “Filter Supplier” prepares a Purchase Order File (PO) for the “MSB” which includes the following xml files:

IMRT Filter Type (IMRT Filters and/or IMRT Radiation Blocking Filters and/or

Radiation Wound Protective Filters)

IMRT Filter Attenuating Material Type (Polyurethane, Brass or Aluminum)

IMRT Filter Label File

“Client” Shipping Label File

IMRT Filter CNC “G-Code” Files; and

IMRT Filter Quality Analysis Files.

Step-9 The “Filter Supplier” then exports the PO to the Manufacturing Service Bureau (MSB);

Step-10 The “MSB” prints the IMRT Filter Label File and attaches the labels to the IMRT Filter Blanks;

Step-11 The “MSB” prints the “Client” Shipping Label File and attaches the Shipping Label/s to the Shipping Carton/s;

Step-12 The “MSB” places the IMRT Filter Blanks in a First-In-First-Out (FIFO) “MSB” Production Queue;

Step-13 The “MSB” places the “Client” Shipping Carton/s in a First-In-First-Out (FIFO) “MSB” Shipping Queue;

Step-14 When a CNC Machine becomes available, the “MSB” Technician uses a Barcode Reader to scan the label of the next IMRT Filter Blank in the FIFO Queue to retrieve the CNC Milling Code from the “Converter Database”, via the secure “MSB” SFTP Server, to mill the Compensator Filter in the Negative for Compensator Molds (made from Polyurethane or Oncology Styrofoam) and in the Positive for Solid Compensators (made from Brass 360 or Aluminum 6061T6);

Step-15 Once the four milling process steps (Rough Cut, Finish Cut, Location Hold Cut and Engraving) are complete, the “MSB” does a Quality Analysis (QA) Test using the 3 Dimensional Computer Numerical Code (CNC “G-Code”) Machine Quality Analysis (QA) Nine-Point Probe Test File to verify the accuracy (acceptable tolerance) of the Compensator Filter prior to removal from the CNC machine;

Step-16 If the acceptable tolerances are met, the “MSB” cleans the IMRT Filter Compensator for shipping;

Step-17 The “MSB” exports the Quality Analysis (QA) Nine-Point Probe Test Results back to the “Filter Supplier” for storage in the “Converter Database”;

Step-18 If the acceptable tolerances are NOT met, the “MSB” destroys the IMRT Filter, does a CNC Machine Inspection to determine the error, makes the necessary correction and prints a new IMRT Filter Label, attaches the label to an IMRT Filter Blank, places the new IMRT Filter Blank in front of the “MSB” Production FIFO Queue;

Step-19 The “MSB” Shipping Clerk uses the Barcode Reader to match the IMRT Filter labels with the correct “Client” Shipping Carton/s label/s and packages the IMRT Filter Compensators with the matching labels for that Cancer Patient and for that “Client”;

Step-20 The “MSB” ships the IMRT Filter Compensators for the Cancer Patient to the “Client” and sends a Tracking/Confirmation Code to the “Filter Supplier” and the “Client”;

Step-21 The “Client” uses a Barcode Reader and retrieves the instructions, via a secure “Client” SFTP Server, to fill the Negative Mold with the exact attenuating material, Tungsten Metal Powdered Metal or molten Cerrobend and how to mount the IMRT Filter on the correct Tray;

Step-22 The “Client” uses a Barcode Reader and retrieves the instructions, via a secure “Client” SFTP Server, for the solid IMRT Filter for the exact attenuating material, Brass 360 or Aluminum 6016T6 and how to mount the IMRT Filter on the correct Tray;

Step-23 The “Client” inserts the IMRT Filter Compensator mounted on the tray using the instructions from the “Converter Database” into the correct accessory holder for the Tray of the accelerator used for calculating the linear attenuating coefficient and does a Dosimetric Film Test;

Step-24 If the Dosimetric Film Test is approved the filled IMRT Filter Compensator Mold or the Solid IMRT Filter Compensator is now ready to be used for the Cancer Patients thirty day treatment cycle;

Step-25 The “Client” sends (exports), via a secure “Client” SFTP Server, the acceptance results to the “Converter Database”;

Step-26 The “Filter Supplier” archives the data for each Cancer Patient's IMRT Filter Compensator(s) within the “Converter Database”; and

Step-27 The “Filter Supplier” informs the “MSB” of the “Client” success.

Turning now to FIG. 1b, there is seen an exemplary implementation of an IMRT Filter Label. The advantage of building a secure “MSB” SFTP Server “Converter Database” using an Automated Programming Interface (API) is when the next “MSB” CNC Machine becomes available, the “MSB” Manufacturing Technician uses a Barcode Reader to scan the label, such as is seen FIG. 1b, of the next IMRT Filter Blank in the “MSB” FIFO Production Queue to retrieve, via a secure “MSB” SFTP Server, the CNC Milling Code Milling Instructions from the “Converter Database” to mill the IMRT Filter Compensator for a specific “Client”, for a specific accelerator, for a specific Attenuating Material, for a specific Tray Location, and for this Patient's specific Treatment Angle, via the secure “MSB” SFTP Server. This method eliminates any possibility of Human Error.

Relative to the IMRT Filter Label seen in FIG. 1b, (xxxx,yyyy) codes are automatically selected from DICOM RT Patient Treatment Plan. The IMRT Filter Label seen in FIG. 1b is automatically constructed from the “Converter Database” for each Intensity Modulated Radiation Filter (IMRT Filter), which corresponds to at least one particular type radiation accelerator treatment machine, having a size requirement, and tray compatibility for the intended radiation accelerator treatment machine. Note the following IMRT Filter Label Barcode values in the following Table X.

TABLE X

| Name | IMRT Filter Label Barcode (Value) | Dicom Tag |
|---|---|---|
| “Client” ID* | aaab cdef ghi xxxz ddmmyy lmno pq jjjkk | |
| | InstitutionName | (0008, 0080) |
| Accelerator Name ID* | aaab cdef ghi xxxz ddmmyy lmno pq jjjkk | |
| | TreatmentMachineName | (300A, 00B2) |
| Accelerator SN ID* | aaab cdef ghi xxxz ddmmyy lmno pq jjjkk | |
| | DeviceSerialNumber | (0018, 1000) |
| Tray Selection* | aaab cdef ghi xxxz ddmmyy lmno pq jjjkk | |
| | d = 1 = Upper Wedge Tray* | (300A, 00DA) |
| | d = 2 = Block Tray* | (300A, 00F6) |
| | d = 3 = Lower Wedge Tray* | (300A, 00E6) |
| Tray Distance* | aaab cdef ghi xxxz ddmmyy lmno pq jjjkk | |
| | SourceToCompensatorTrayDistance: | (300A, 00E6) |
| Mounting IMRT Filter* | aaab cdef ghi xxxz ddmmyy lmno pq jjjkk | |
| | CompensatorMountingPosition* | (300A, 02E1) |
| | f = 1 = Radiation Source Side* | |
| | f = 2 = Patient Side* | |
| Filter Type* | aaab cdef ghi xxxz ddmmyy lmno pq jjjkk | (300A, 00F8) |
| | g = 1 = IMRT Filter | |
| | g = 2 = IMRT Radiation Blocking Filter | |
| | g = 3 = Radiation Wound Protective Filter | |
| IMRT Material ID* | aaab cdef ghi xxxz ddmmyy lmno pq jjjkk | |
| | MaterialID* | (300A, 00E1) |
| | h = 1 = Tungsten Powdered Metal | |
| | h = 2 = Molten Cerrobend | |
| | h = 3 = Aluminum (6061T6) | |
| | h = 4 = Brass 360 | |
| | h = 5 = Custom | |
| Number of Filters* | aaab cdef ghi xxxz ddmmyy lmno pq jjjkk | |
| | NumberOfCompensators* | (300A, 00E0) |
| | i = 0 = 10 IMRT Filters | |
| | i Max = 10 | |
| | i Min = 3 | |
| Patient ID#* | aaab cdef ghi xxxz ddmmyy lmno pq jjjkk | (0010, 0020) |
| | z = 1 = PatientName* | (0010, 0010) |
| | z = 2 = PatientSex* | (0010, 0040) |
| | z = 3 = PatientBirthDate* | (0010, 0030) |
| Patient Plan Date* | aaab cdef ghi xxxz ddmmyy lmno pq jjjkk | |
| | StudyDate* | (0008, 0020) |
| | StudyTime* | (0008, 0030) |
| Fluence Plan Number* | aaab cdef ghi xxxz ddmmyy lmno pq jjjkk | |
| | l = ReferencedBeamNumber* | (300C, 0006) |
| | m = BeamMeterset* | (300A, 0086) |
| | n = NumberOfBeams* | (300A, 0080) |
| | o = BeamSequence* | (300A, 00B0) |
| Fluence Plan Field Number* | aaab cdef ghi xxxz ddmmyy lmno pq jjjkk | |
| | p = BeamName* | (300A, 00C2) |
| | q = BeamNumber* | (300A, 00C0) |
| “MSB” ID** | aaab cdef ghi xxxz ddmmyy lmno pq jjjkk | |
| “MSB” Quantity of CNC** | aaab cdef ghi xxxz ddmmyy lmno pq jjjkk | |
| | kk = Number of CNC Machines | |

*Taken automatically from DICOM RT Patient Treatment Plan and Converter Database
**Taken automatically from Converter Database

By using an Automated Programming Interface (API) and the Barcode Reader, the tooling instructions within the “Converter Database” are selected automatically for the “Rough Cut” tool removal of material to form the IMRT Filter in the tooling database comprises at least one tool, a ¼″ ball-end-mill, associated therewith to be used by the automated CNC machine. This method eliminates any possibility of Human Error.

By using an API and the Barcode Reader, the tooling instructions within the “Converter Database” are selected automatically for the “Finish Cut” tool removal of material to form the IMRT Filter in the tooling database comprises at least one tool, a ⅛″ ball-end-mill, associated therewith to be used by the automated CNC machine. This method eliminates any possibility of Human Error.

By using an API and the Barcode Reader, the tooling instructions within the “Converter Database” are selected automatically for the “Location Hole” tool removal of material to form the IMRT Filter in the tooling database comprises at least one tool, a Letter F Drill, associated therewith to be used by the automated CNC machine. This method eliminates any possibility of Human Error.

By using an Automated Programming Interface (API) and the Barcode Reader, the tooling instructions within the “Converter Database” are selected automatically for the “Engraving” tool removal of material to form the IMRT Filter in the tooling database comprises at least one tool, an Engraving Tool, associated therewith to be used by the automated CNC machine. This method eliminates any possibility of Human Error.

By using an API and the Barcode Reader, the tooling instructions within the “Converter Database” are selected automatically for the “Tolerance Calibration” tool to test the depth of cut at selected XY locations within the IMRT Filter in the tooling database comprises at least one tool, an “Inspection Probe”, associated therewith to be used by the automated CNC machine. This method eliminates any possibility of Human Error.

Each set of tooling instructions are defined based upon the 3 Dimensional XYZ data converted from the specific “Client” DICOM RT Cancer Patient Treatment Plan and stored within the “Converter Database” using an Automated Programming Interface (API). This method includes the automated conversion of the optimal fluence field data for each Cancer Patient Treatment Field angle into a 3 Dimensional Computer Numerical Code (CNC) Machine Control Files to be used to automatically control the milling of the Intensity Modulated Radiation Filter; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and for a specific Linear Attenuation Coefficient that is used for each Cancer Patient's treatment angle. This CNC Machine Control File is sent (exported) via the Internet using a secure “MSB” SFTP Server to the Manufacturing Service Bureau (MSB) using an Automated Programming Interface (API) for manufacturing the Cancer Patient's Set of Intensity Modulated Radiation Filters (IMRT Filters); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and for a specific Linear Attenuation Coefficient.

Each set of tooling instructions are defined based upon the 3 Dimensional XYZ data converted from the 3 Dimensional Compensator Files received (imported) from “Clients” that have Treatment Planning Systems that can produce Compensator Files via the Internet using a secure “Client” SFTP Server and stored within the “Converter Database” using an Automated Programming Interface (API). This method includes the automatic conversion into 3 Dimensional Computer Numerical Code (CNC) Machine Control Files to be used to automatically control the milling of the Intensity Modulated Radiation Filter; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and for specific Linear Attenuation Coefficient used for each Cancer Patient's treatment angle. This CNC Machine Control File is sent (exported) via the Internet using a secure “MSB” SFTP Server to the Manufacturing Service Bureau (MSB) using an Automated Programming Interface (API) for manufacturing the Cancer Patient's Set of Intensity Modulated Radiation Filters; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and a specific Linear Attenuation Coefficient.

Each set of tooling instructions stored within the “Converter Database” using an API further comprises the XY specific hole locations in relationship to the Isocenter for the specific radiation accelerator for the four holes to be drilled through the IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and a specific Linear Attenuation Coefficient, wherein the foregoing are automatically taken from the “Converter Database” for that “Client”, for that Accelerator, for that Tray location, and for that attenuating material.

Each set of tooling instructions stored within the “Converter Database” using an API further comprises engraving the Isocenter X1 X2 and Y1 Y2 in relationship to the Isocenter for the specific radiation accelerator taken from the “Converter Database” on the source side surface of the Intensity Modulated Radiation Filter; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, for a specific tray location, and for a specific Linear Attenuation Coefficient; wherein the foregoing are automatically taken from the “Converter Database” for that “Client”, for that Accelerator, for that Tray location, and for that attenuating material.

Each set of tooling instructions stored within the “Converter Database” using an API further comprises engraving the CNC Machine Identification Number assigned, based on the “Converter Database” on the source side surface X2 Y1 quadrant of the Intensity Modulated Radiation Filter; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient; wherein the foregoing are automatically taken from the “Converter Database” for that “Client”, for that Accelerator, for that Tray location, and for that attenuating material.

Each set of tooling instructions stored within the “Converter Database” using an API for the measuring of the Z value for nine inspection points on the Intensity Modulated Radiation Filter (IMRT Filter) after machining; defined within the inspection database based upon the Nine-Point Probe File measured inspection points and the received converted DICOM RT Cancer Patient Treatment Plan Field CNC (“G-Code”) Data for each IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and a specific Linear Attenuation Coefficient; and generating a quality assurance report based upon the defined inspection database for that “Client”, for that Accelerator, for that Tray location, and for that attenuating material.

Each set of tooling instructions stored within the “Converter Database” uses an API for the acceptable tolerances that are matched to an acceptable tolerance automatically. If the tolerance is within acceptable limits, the IMRT Filter Compensator is cleaned and placed at the end of the Shipping Queue.

Each set of tooling instructions stored within the “Converter Database” using an API for the acceptable tolerances are matched to an acceptable tolerance automatically. If the tolerance is NOT within acceptable limits, the IMRT Filter Compensator is destroyed and a new Filter Label is generated, attached to the IMRT Filter Blank and placed in front of the FIFO Production Queue for milling.

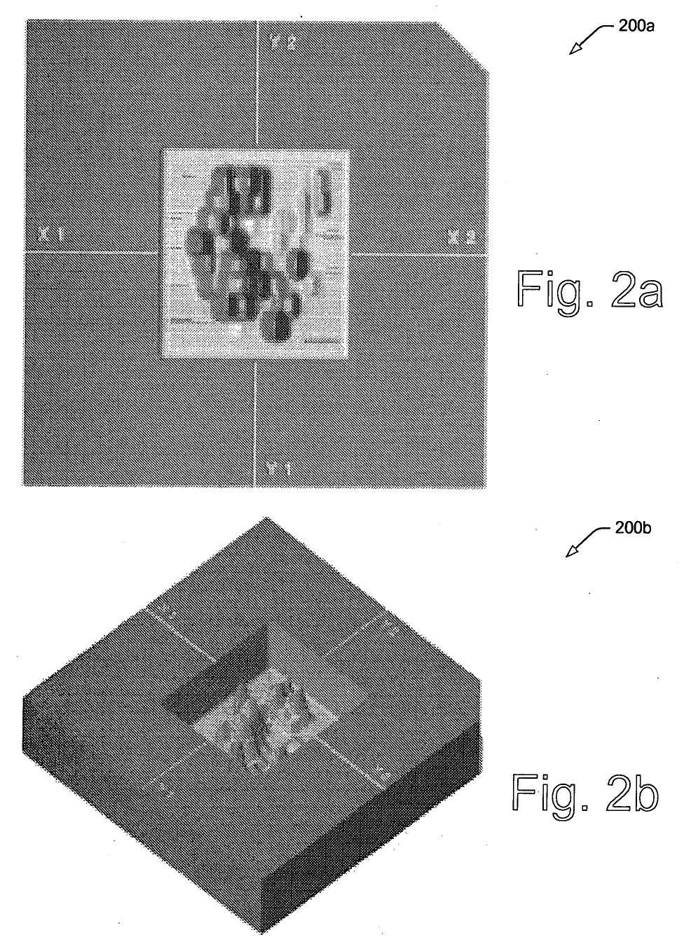

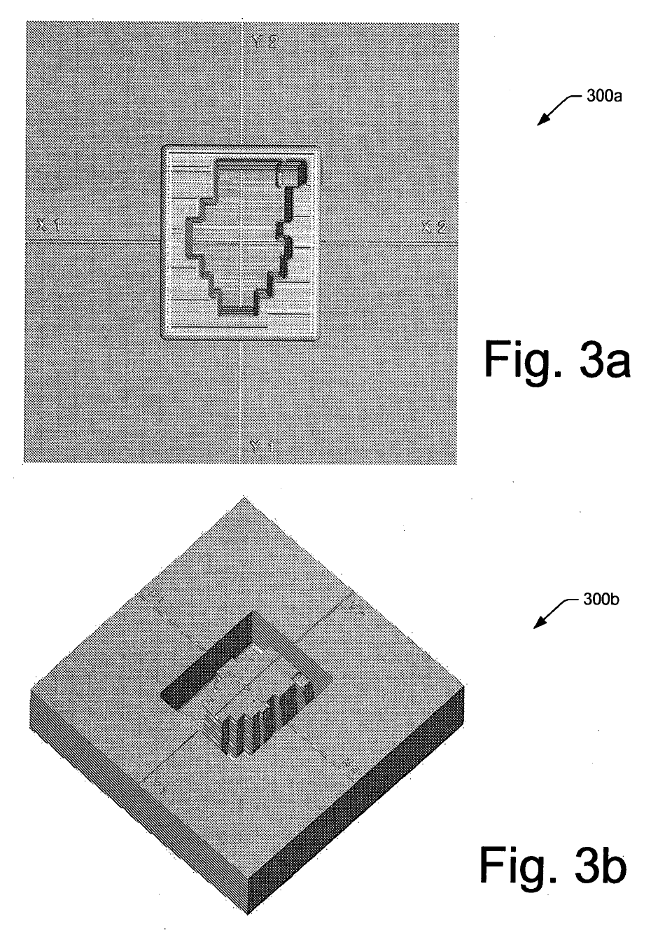

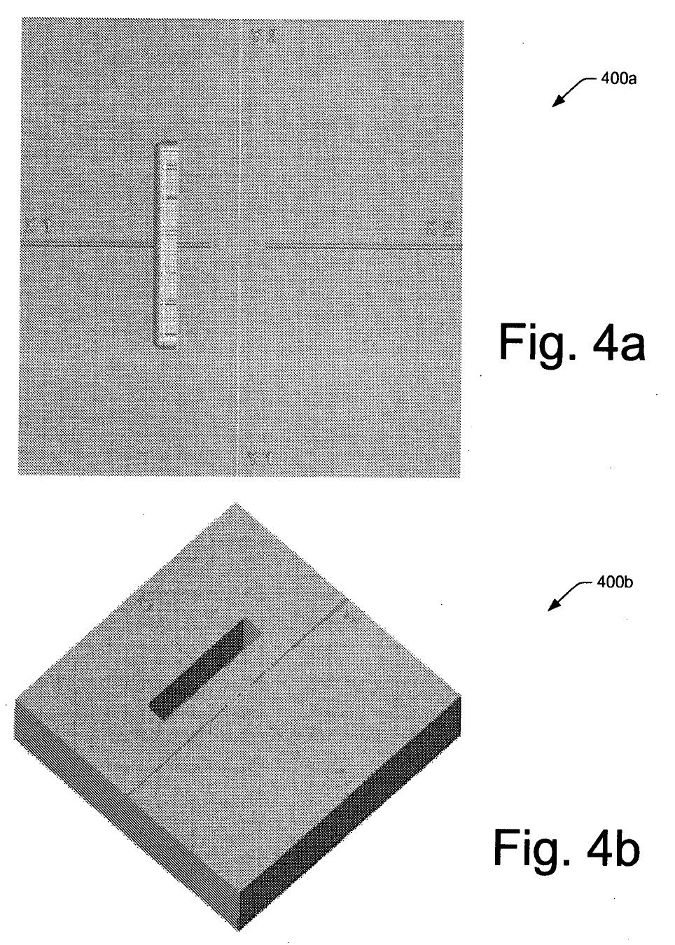

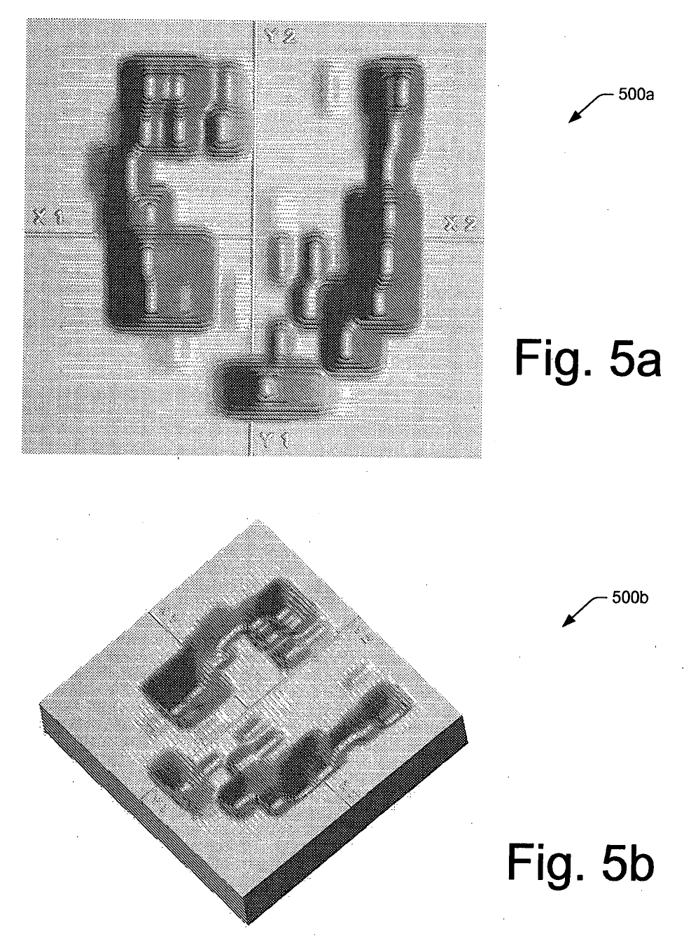

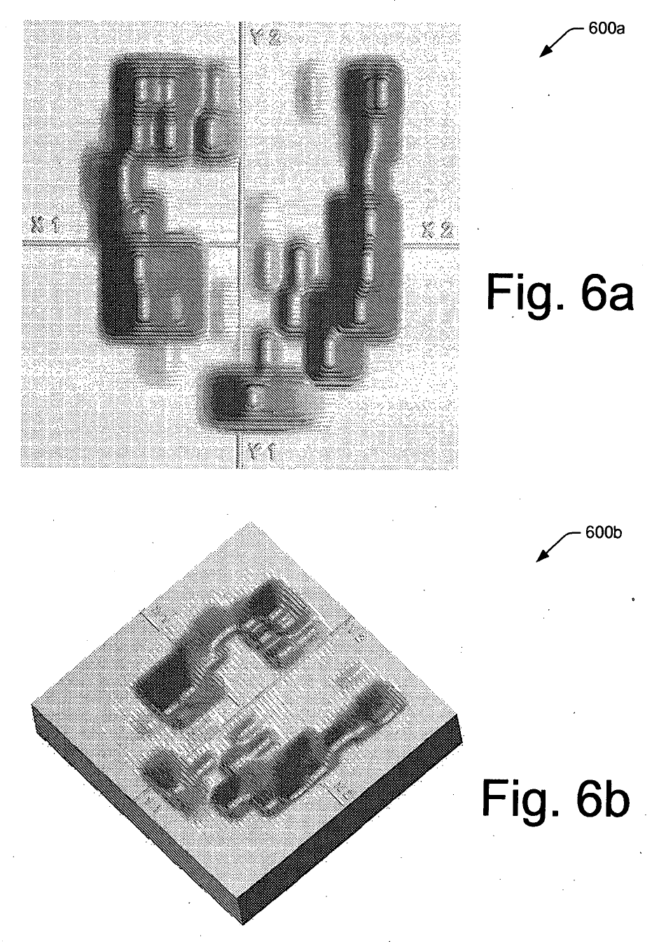

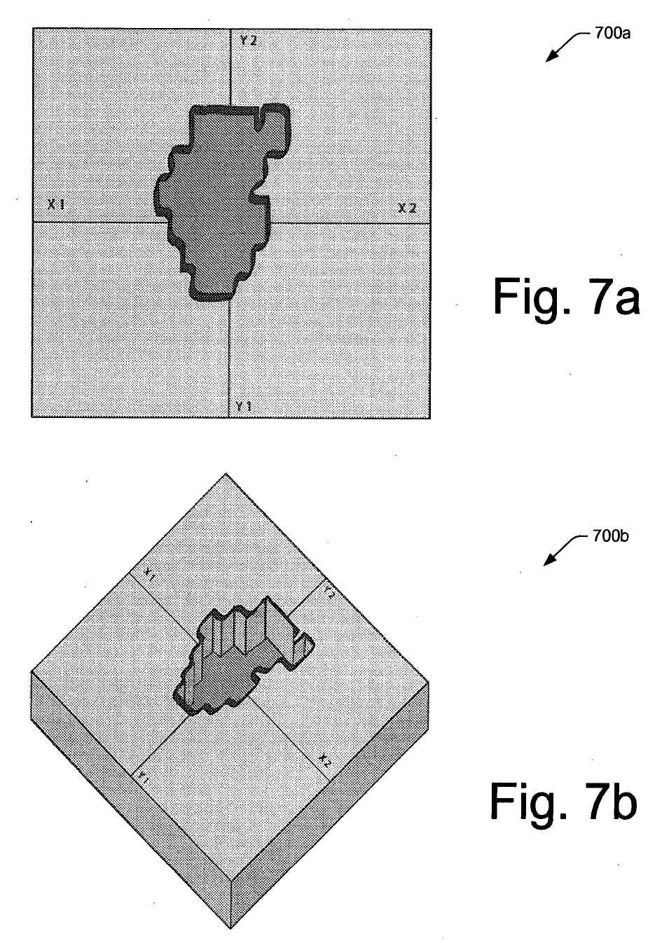

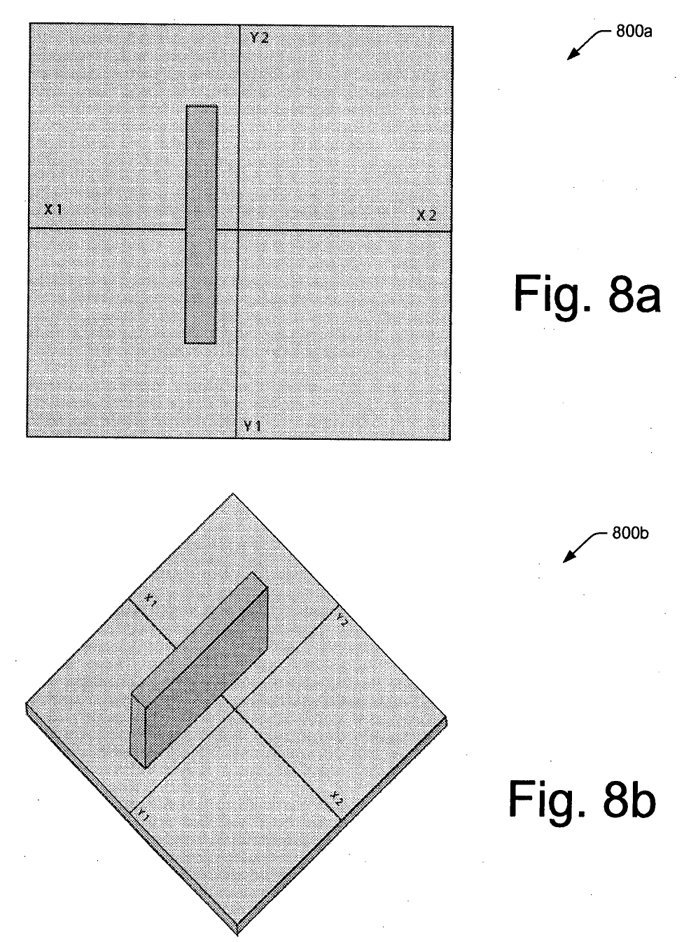

FIGS. 2a-2b depicts an implementation of a Compensator Negative Oncology Styrofoam or Polyurethane IMRT Filter Negative Mold illustrated, respectively, in isometric and plan views. FIGS. 3a-3b depict, respectively, isometric and plan views of an implementation of an Intensity Modulated Radiation Blocking Filter Negative Mold to be used in a radiation accelerator treatment machine. FIGS. 4a-4b depict, respectively, isometric and plan views of an implementation of an Intensity Modulated Radiation Wound Protective Filter Negative Mold to be used in a radiation accelerator treatment machine. FIGS. 5a-5b depict, respectively, isometric and plan views of an implementation of an IMRT Filter (Compensator) milled from solid Brass 360 to be used in a radiation accelerator treatment machine. FIGS. 6a-6b depict, respectively, isometric and plan views of an implementation of an IMRT Filter (Compensator) milled from solid Aluminum 6061T6 to be used in a radiation accelerator treatment machine. FIGS. 7a-7b depict, respectively, isometric and plan views of an implementation of an IMRT Radiation Blocking Solid Filter to be used in a radiation accelerator treatment machine. FIGS. 8a-8b depict, respectively, isometric and plan views of an implementation of an IMRT Radiation Wound Protection Solid Filter to be used in a radiation accelerator treatment machine.

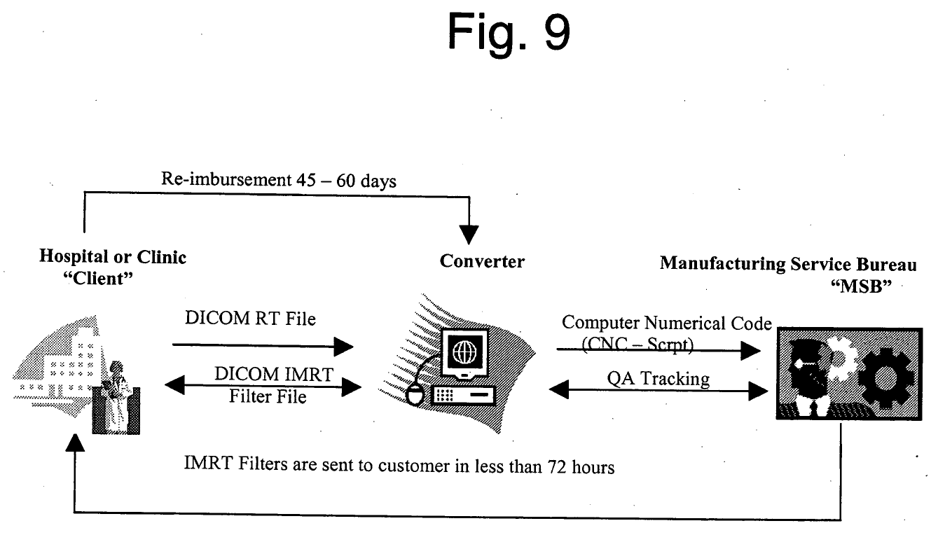

FIG. 9 depicts, in a block level diagram, three distinct implementations of process flows among and between (i) Client; (ii) A Converter; and (iii) MSB. Other Figures include a reference to “Innocure”. The reference to “Innocure” in the Figures is an exemplary trade name descriptive of the Converter seen in FIG. 9.

The first implementation is the Radiation Oncology Hospital or Clinic (“Client”) who provides the Digital Imaging and Communications in Medicine Radiation Therapy (DICOM RT) Cancer Patient Treatment Plan File or the 3 Dimensional Compensator Files for conversion. The second is the ‘Converter’ who provides Conversion Software Device to convert the Digital Imaging and Communications in Medicine Radiation Therapy (DICOM RT) and/or 3 Dimensional Compensator Files into high resolution IMRT Filters (Compensators) Computer Numerical Code (CNC) for manufacturing (milling). The third is the Manufacturing Service Bureau (“MSB”) who manufactures the IMRT Filters.

One implementation includes for a Radiation Hospital or Clinic (“Client”) providing the Digital Imaging and Communications in a Medicine Radiation Therapy (DICOM RT) Cancer Patient Treatment Plan File or the 3 Dimensional Compensator Files for conversion. The “Client” also provides Patient Care by using the Intensity Modulated Radiation Filters (IMRT Filters); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient.

A method for building a secure “Client” SFTP Server “Converter Database” by a “Commissioning Process”, which includes; establishing a “Client” ID, “Client” Address, “Client” Contact Information, “Client” Password, “Client” Accelerator ID, selection of an Attenuating material for the “Client”, Linear Attenuation Coefficient (LAC) calculated for that material and specific radiation accelerator treatment machine, the Tray selected for mounting the IMRT Filter, Upper Wedge Tray Distance from Source, Wedge Tray Thickness, Block Tray Distance from Source, Block Tray Thickness, Lower Wedge Tray Distance from Source, Lower Wedge Tray Thickness, the Compensating Filter Tray mounting position (source side of selected Tray or patient side of selected Tray), Isocenter Distance from Source, Isocenter Calibration Markings, X1 X2 and Y1 Y2, on the Upper Wedge Tray, the Block Tray, the Lower Wedge Tray, the “Client” Attenuating Material selected and the “Client” Linear Attenuation Coefficient calculated for that “Client” Accelerator ID.

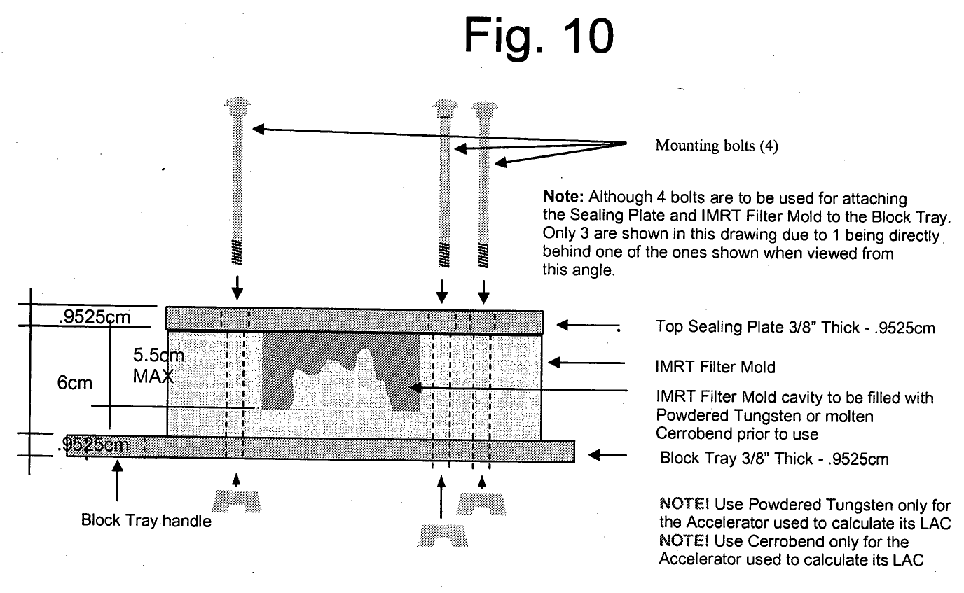

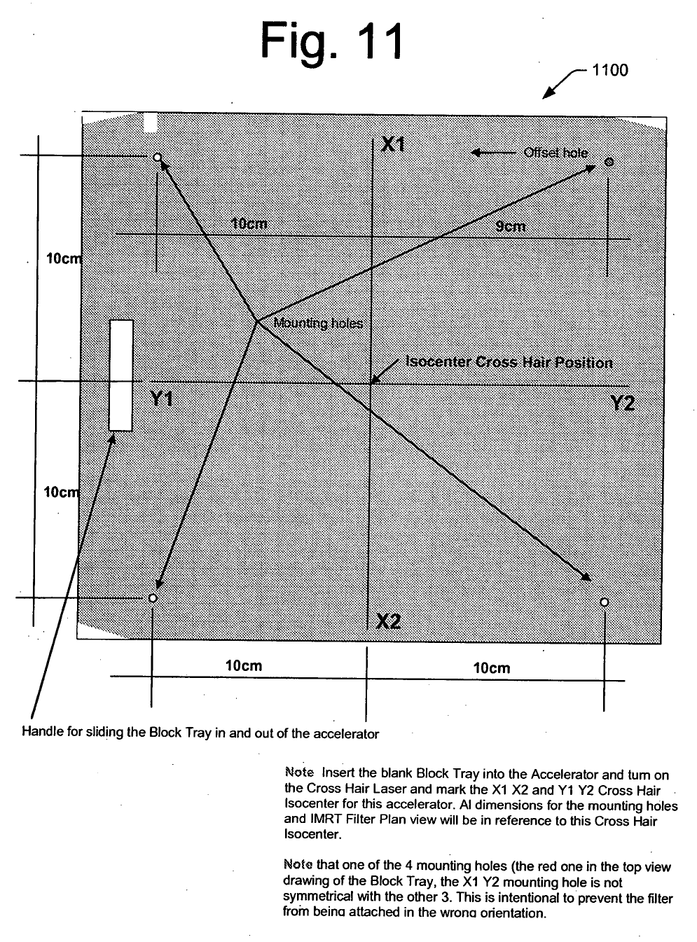

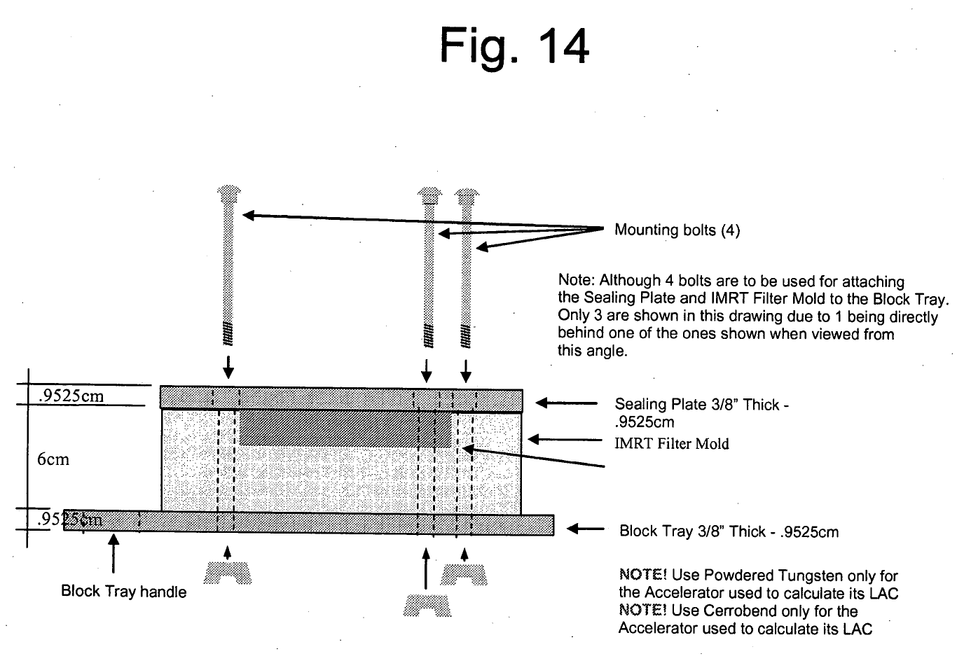

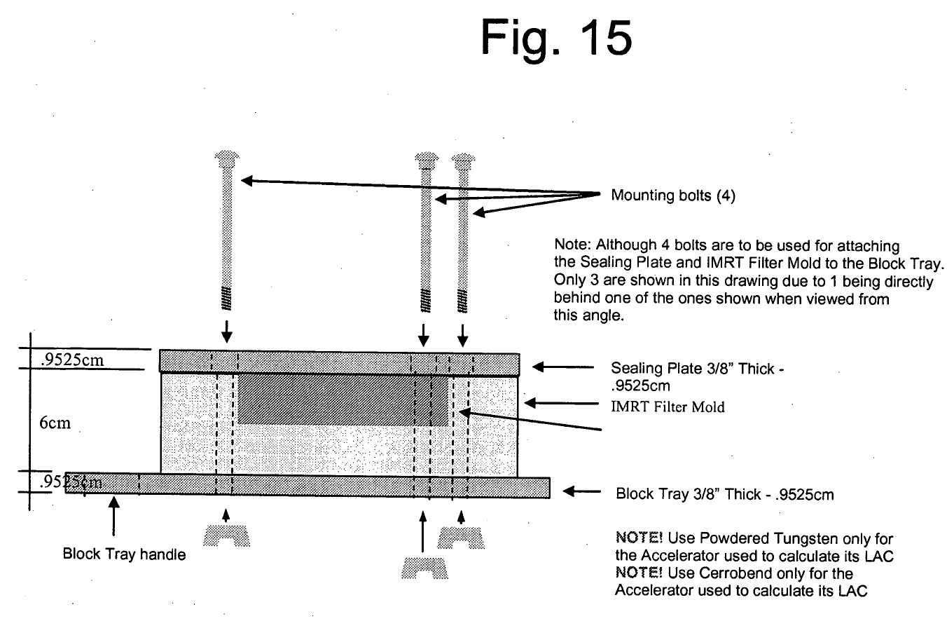

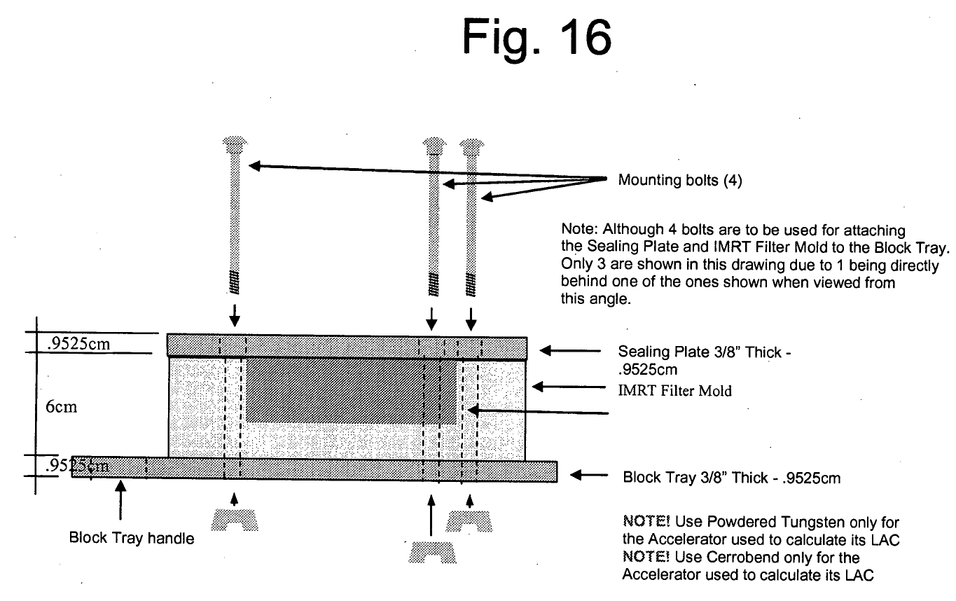

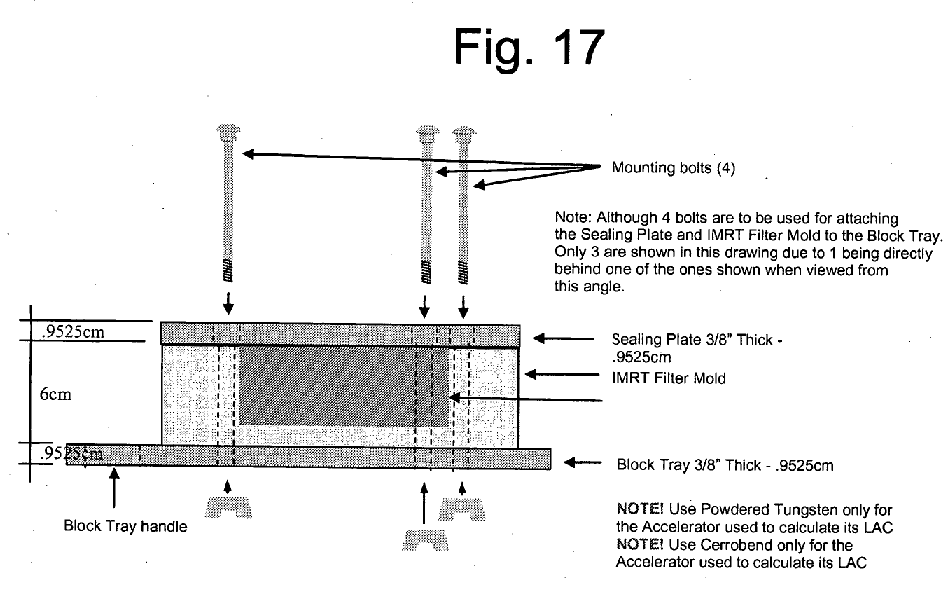

A method can be used to calculate the Linear Attenuation Coefficient (LAC) for the selected Tungsten Metal Powder attenuating material (see FIGS. 10 through 17) and for a specific radiation accelerator treatment machine for the actual Tray selected for mounting the IMRT Filter by using five (5) Negative Test Molds with a particular IMRT Filter Mold cavity having dimensions of 12.5 cm×12.5 cm×1 cm depth, ×2 cm depth, ×3 cm depth, ×4 cm depth and ×5 cm depth, where the cavity is to be filled with Attenuating material with the Tungsten Powder. This same method can be used to do the calculations for Linear Attenuation Coefficient (LAC) of Cerrobend.

A method can also be used to calculate the Isocenter Distance from Source, Isocenter Calibration Markings, X1 X2 and Y1 Y2, on the selected Tray for mounting the IMRT Filter.

FIG. 10 depicts a side view of a Block Tray with an IMRT Filter Mold and a Top Sealing Plate.

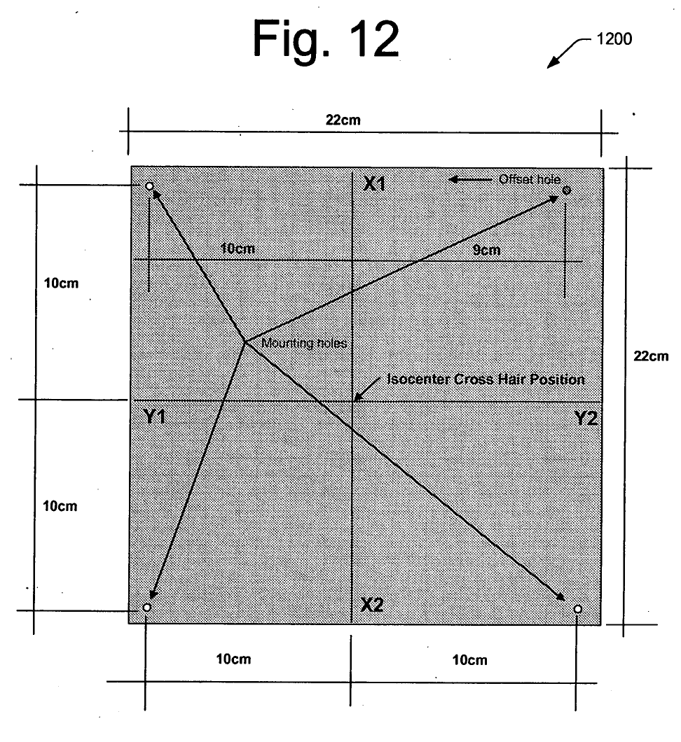

A method can be used for a set of four reference holes, which have to be correlated to the Accelerator Plan Field Laser Cross Hair XY Origin (Isocenter), which have been milled in the Negative Mold to match the four holes milled in the Selected Mounting Tray (Polycarbonate) and Top Sealing Plate (Polycarbonate). The location, diameter, and depth of the reference holes are fixed in relation to the Accelerator Plan Field Cross Hair XY Origin (Isocenter) are used to eliminate the possibility of an IMRT Filter to be mounted incorrectly (see FIGS. 11 and 12).

FIG. 11 depicts a Plan view of a Polycarbonate BLOCK Tray with Mounting Holes & Dimensions, and FIG. 12 depicts a Plan view of a Polycarbonate Top Sealing Plate with Holes & Dimensions.

A method can be used in the commissioning process to inscribe or mark the Plan Field Cross Hairs (Isocenter X1 X2 and Y1 Y2) on the top of the Selected Mounting Tray (Polycarbonate) once it is inserted into the Accelerator to collect the Linear Attenuation Coefficient (LAC) of that Accelerator. This Isocenter X1 X2 and Y1 Y2 will be used by the Conversion Software to reference the origin of all DICOM RT Patient (.dcm) treatment plan Optimal Fluence Field files using the “DICOM2IMRTFilterCompensatorConverter” for this specific Accelerator. The LACs collected for the Selected Mounting Tray (Polycarbonate), the Top Sealing Plate (Polycarbonate) and the Tungsten Powder or Cerrobend will also be stored in the Converter/Hospital Client Database.

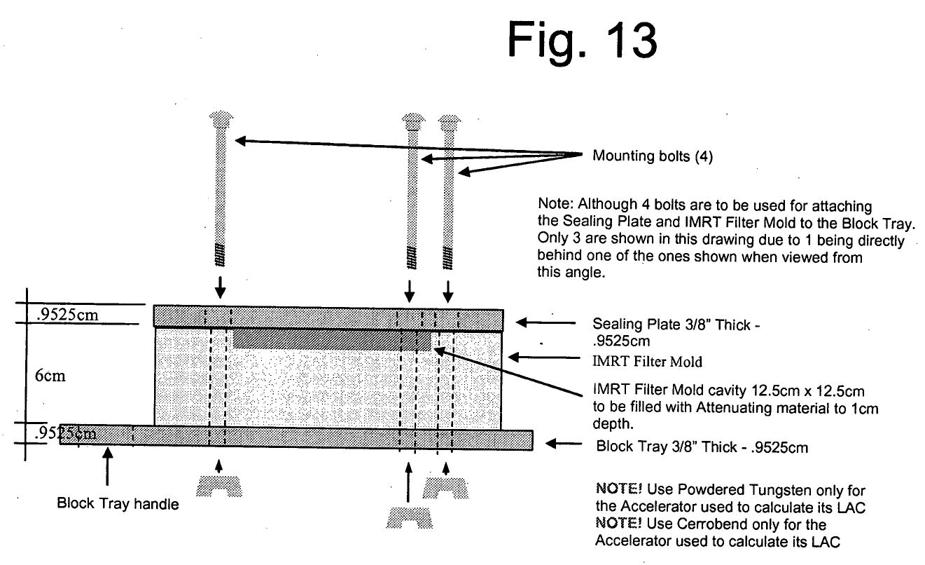

FIGS. 13-17 depict respective side views of a Block Tray with an IMRT Filter Mold and the Top Sealing Plate with five (5) multiple depths, used to test the linear attenuation coefficient of the selected attenuating material during the Commissioning Process.

Each type of radiation accelerator treatment machine has a Hospital Loading and Testing Requirement for each Intensity Modulated Radiation Filter (IMRT Filter); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient. This Loading and Testing Requirement is accessed automatically by using a Barcode Reader and the “Converter Database” for automatically selecting the Instructions for loading and testing each IMRT Filter, Negative Mold IMRT Filters or Solid IMRT Filters, for the intended radiation accelerator treatment machine, prior to Patient Use.

The “Client” receives the Carton of Negative Mold IMRT Filters and uses a Barcode Reader on each IMRT Filter, which automatically accesses the “Converter Database” via the Internet using a secure “Client” SFTP Server and retrieves the instruction information on how to fill the Negative Mold for the IMRT Filter and/or IMRT Radiation Blocking Filter used for each Cancer Patient's Treatment Field angle; and/or Radiation Wound Protective Blocking Filter used for each Cancer Patient's Surgical Incision, for a specific accelerator, with the exact attenuating material, Tungsten Metal Powdered Metal or molten Cerrobend, and how to mount the IMRT Filter on the correct Tray.

The “Client” receives the Carton of Solid IMRT Filters and uses a Barcode Reader on each IMRT Filter, which automatically accesses the “Converter Database” via the Internet using a secure “Client” SFTP Server and retrieves the instruction information to fill the Solid IMRT Filter for the IMRT Filter and/or IMRT Radiation Blocking Filter used for each Cancer Patient's Treatment Field angle; and/or Radiation Wound Protective Blocking Filter used for each Cancer Patient's Surgical Incision, for a specific accelerator, with the exact attenuating material, Brass 360 or Aluminum 6016T6, and how to mount the IMRT Filter on the correct Tray.

The “Client” inserts the Compensator Filter mounted on the tray using the instructions from the “Converter Database” into the accessory holder of the accelerator for that Tray and does a Dosimetric Film Test.

The “Client” Dosimetric Film Test is approved by the Radiation Oncologist, the filled Negative Mold IMRT Filter or the Solid IMRT Filter is now ready to be used for the Cancer Patient's thirty day treatment cycle. The set of Patient IMRT Filters are stored in the “Client” wall mounted IMRT Filter Storage Rack with the IMRT Filter Barcode Labels visible.

The “Client” sends (exports) the acceptance results to the “Converter Database”, via the secure “Client” SFTP Server.

The Converter archives the data for each Cancer Patient's IMRT Filter and/or IMRT Radiation Blocking Filter used for each Cancer Patient's Treatment Field angle; and/or Radiation Wound Protective Blocking Filter used for each Cancer Patient's Surgical Incision, for a specific accelerator, for a specific Radiation Oncology Hospital or Clinic “Client”, with the exact attenuating material, within the “Converter Database”.

The “Client” prepares a Patient Barcode Wrist Band, using the Converter Database, for the Patient's thirty day treatment. This feature advantageously reduces employee intervention by using a Barcode Reader to automatically select the correct IMRT Filter for the intended radiation accelerator treatment machine for the correct Patient for the correct Treatment Angle by matching the Patient Wrist Band with the IMRT Filters.

The “Client” prepares the Patient for the normal thirty days of treatment, five days a week over six weeks by attaching a Wrist Band with Bar-coded information that matches the “Client” Treatment Planning Database and the Converter Database. This Wrist Band will be worn by the Patient for the thirty days of treatment.

The Patient arrives at the “Client” location for his/her radiation treatment. The Radiotherapist assists the Patient to lie down on the treatment table. Selects the first IMRT Filter by Patient's Name from the IMRT Filter Storage Rack and inserts the IMRT Filter into the radiation accelerator accessory Tray Slot. The Barcode Reader is first used to determine if this IMRT Filter matches the Patient's wrist band. If automatically approved, the second step is allowed to verify that the IMRT Filter is placed in the correct Tray Slot. If automatically approved, the third step is allowed to automatically rotate the radiation accelerator treatment machine to the correct angle for this Patient's Treatment Field for this IMRT Filter. The Radiotherapist leaves the treatment room and the radiation dose is delivered automatically. The “Converter Database” is automatically updated for this IMRT Filter for this specific treatment angle with the treatment date. This IMRT Filter cannot be used twice on same day eliminating any possible Human Error.

The Radiotherapist enters the treatment room and selects the next IMRT Filter by the Patient's Name from the IMRT Filter Storage Rack and inserts the IMRT Filter into the radiation accelerator accessory Tray Slot. The Barcode Reader is again used to automatically determine if this IMRT Filter matches the Patient's wrist band. If automatically approved, the next step is to automatically verify that the IMRT Filter is placed in the correct Tray Slot. If automatically approved, the next step is to automatically rotate the radiation accelerator treatment machine to the correct angle for this Patient's Treatment Field. The Radiotherapist leaves the treatment room and the radiation dose is delivered automatically. The “Converter Database” is automatically updated for this IMRT Filter for this specific treatment angle with the treatment date. This IMRT Filter cannot be used twice on same day eliminating any possible Human Error.

The above steps are repeated until all the IMRT Filters for this Patient have been accessed on this Treatment Date. The Radiotherapist cannot treat another patient until the “Converter Database” automatically approves the completion of this Patient's Treatment for this Date. This also eliminates any possible Human Error.

The above treatment daily cycle process is repeated for this Patient five days a week for the total of six weeks, thirty treatment plan days. The Radiotherapist cannot treat this patient for a thirty first day because the “Converter Database” automatically locks this Patient's Treatment Plan Files when the last Treatment Date is finished. This also eliminates any possible Human Error.

The second embodiment in FIG. 9 is the Converter. The Converter provides the conversion software device, “DICOM2IMRTFilterCompensatorConverter”, to convert the Digital Imaging and Communications in Medicine Radiation Therapy (DICOM RT) and/or 3 Dimensional Compensator Files into IMRT Filters (Compensators) Computer Numerical Code (CNC) for manufacturing (milling).

Referring initially to FIGS. 1b, 2a-2b, 3a-3b. and 4a-4b, implementation are directed to methods for providing an Intensity Modulated Radiation Filter (IMRT Filter); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and a specific Linear Attenuation Coefficient. The illustrated Intensity Modulated Radiation Filter (IMRT Filter) is also known as a compensator or as a modulator, and is machined from an Oncology Styrofoam or Polyurethane Foam material. This foam material may be referred to as an Intensity Modulated Radiation Filter (IMRT Filter) blank.

Referring initially to FIGS. 5a-5b, 6a-6b, 7a-7b and 8a-8b, implementations are directed to methods for providing an Intensity Modulated Radiation Filter (IMRT Filter); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and a specific Linear Attenuation Coefficient. The illustrated Intensity Modulated Radiation Filter (IMRT Filter) is also known as a compensator or as a modulator, and is machined from a solid piece of material. This solid piece of material may be Aluminum 6061T6 or Brass 360, for example, and is commonly referred to as an Intensity Modulated Radiation Filter (IMRT Filter) blank.

As discussed above, Radiation Oncology Treatment Planning Systems can provide either; a Digital Imaging and Communications in Medicine Radiation Therapy (DICOM RT) Cancer Patient Treatment Plan File for export; or a set of 3 Dimensional Compensator Files for export. Both export files contain the measurement technology at radiation treatment planning centers for determining the precise three-dimensional coordinates for a cancerous tumor along with precise locations and densities for the healthy bone and tissue surrounding it.

Implementations can covert the DICOM RT Cancer Patient Treatment Plan Data File provided by the “Client” and first, automatically produce the multiple Optimal Fluence Field 2-D Matrix Files for each Patient Treatment Angle with an XY grid of 2.5 mm or 5.0 mm spacing (or any grid dimension) produced by the “Client” Treatment Planning System. This grid dimension depends on the leaf thickness of the specific Radiation Accelerator treatment machine and Treatment Planning System used and is automatically selected from the Converter Database. Second, the Optimal Fluence Field 2-D Matrix File for each Patient Treatment Angle is then automatically converted by the Converter into a higher accuracy 3 Dimensional IMRT Matrix File with an XY grid spacing of 0.5 mm or 0.1 mm depending on the selection by the “Client” Radiation Oncologist. Third, the 3 Dimensional IMRT Matrix Files are automatically converted into a DICOM RT Cancer Patient Treatment Plan File with IMRT Filters (Compensators) with Thickness Matrices and Transmission Matrices in the DICOM format for return (export) to the Radiation Oncology Hospital or Clinic (e.g.; “Client”) via the Internet using a secure “Client” SFTP Server for “Dose” Calculations. Fourth, the 3 Dimensional IMRT Matrix Files are automatically converted into the CNC Code 3 Dimensional Milling Instructions to mill each of the Intensity Modulated Radiation Filters (IMRT Filters) for a radiation accelerator treatment machine that uses variations in the thickness of the attenuating material to vary the intensity of the radiation, thus producing a radiation field conforming to the topography of the Cancer Patient.

Implementations can covert the 3 Dimensional Compensator File Data provided by the “Client” with an XY grid of 2.5 mm or 5.0 mm spacing (or any grid dimension) produced by the “Client” Treatment Planning System depending on the leaf thickness of the specific radiation accelerator treatment machine. The Grid Spacing is dependant on the Radiation Accelerator and Treatment Planning System Used and is automatically selected from the “Converter Database” and first, automatically convert the 3 Dimensional Compensator Files into a higher accuracy 3 Dimensional IMRT Matrix File with an XY grid spacing of 0.5 mm or 0.1 mm depending on the selection by the “Client” Radiation Oncologist. Second, automatically convert the higher accuracy XY Coordinates into the CNC Code 3 Dimensional Milling Instructions to mill an Intensity Modulated Radiation Filter (IMRT Filter); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient; that uses variations in the thickness of the attenuating material to vary the intensity of the radiation, thus producing a radiation field conforming to the topography of the Cancer Patient.

Referring now to FIG. 1, the depicted process flow chart illustrates exemplary methods for providing an Intensity Modulated Radiation Filter (IMRT Filter); an IMRT Filter and/or an IMRT Radiation Blocking Filter used for each Cancer Patient's Treatment Field angle; and/or a Radiation Wound Protective Blocking Filter used for each Cancer Patient's Surgical Incision, for a specific Radiation Oncology Hospital or Clinic “Client”, for a specific radiation accelerator treatment machine, a specific tray location, and a specific Linear Attenuation Coefficient. Implementations receive (import) via the Internet, using a secure “Client” SFTP Server, a Digital Imaging and Communications in Medicine Radiation Therapy (DICOM RT) Cancer Patient Treatment Plan File. This file is converted into multiple treatment Optimal Fluence Field 2D Matrices, into multiple IMRT Filter (Compensator) 3 Dimensional Matrices, and into multiple 3 Dimensional Computer Numerical Code (CNC) Files. Thereafter, there is provided a set of Intensity Modulated Radiation Filters (IMRT Filters); IMRT Filters and/or IMRT Radiation Blocking Filters used for each Cancer Patient's Treatment Field angle; and/or Radiation Wound Protective Blocking Filters used for each Cancer Patient's Surgical Incision, for a specific Radiation Oncology Hospital or Clinic “Client”, for a specific radiation accelerator treatment machine, a specific tray location, and a specific Linear Attenuation Coefficient.

The third embodiment of FIG. 9 is the Manufacturing Service Bureau (“MSB”) who manufactures the IMRT Filters.

The tooling instructions are automatically received from the “Converter Database” via the secure “MSB” SFTP Server by the “MSB” for the selected Intensity Modulated Radiation Filter (IMRT Filter) Blank; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient.

This feature advantageously reduces employee intervention by allowing the “MSB” Technician to use a Barcode Reader and the “Converter Database” for automatically selecting the Instructions for Milling the IMRT Filter for the intended radiation accelerator treatment machine. The tooling machine may be a 3-axis or 5-axis milling machine. The cutting tools used by milling machines are commonly known as ball-end-mills. Example milling machines include the FANUC 3-Axis or FANUC 5-Axis milling machines with live automated tooling (other CNC Manufactures are supported as well).

Each tooling strategy for the “Rough Cut” removal of material in the “Converter Database” for this “MSB” tooling instructions comprises at least one, ¼″ Diameter Ball-End-Mill Tool associated therewith to be used by the tooling machine. Each tooling strategy is defined based upon the conversion of a Digital Imaging and Communications in Medicine Radiation Therapy (DICOM RT) Cancer Patient Treatment Plan into multiple Treatment Field 2D Matrices and into multiple IMRT Filter (Compensator) 3 Dimensional Matrices and into 3 Dimensional Computer Numerical Code (CNC) Files and providing a set of Intensity Modulated Radiation Filters (IMRT Filters); IMRT Filters and/or IMRT Radiation Blocking Filters used for each Cancer Patient's Treatment Field angle; and/or Radiation Wound Protective Blocking Filters used for each Cancer Patient's Surgical Incision, to be used by the Radiation Oncology Hospital or Clinic (e.g.; a “Client”) to modulate the radiation dose for the Cancer Patient for a specific radiation accelerator treatment machine.

The strategy for each “Rough Cut” tool is automatically matched within the “Converter Database”; to the “Client” ID, “Client” Address, “Client” Contact Information, “Client” Accelerator ID, selection of an Attenuating material for the “Client”, Linear Attenuation Coefficient (LAC) calculated for that material and specific radiation accelerator treatment machine, the Tray selected for mounting the IMRT Filter, Upper Wedge Tray Distance from Source, Wedge Tray Thickness, Block Tray Distance from Source, Block Tray Thickness, Lower Wedge Tray Distance from Source, Lower Wedge Tray Thickness, Isocenter Distance from Source, Isocenter Calibration Markings, X1 X2 and Y1 Y2, on the Upper Wedge Tray, the Block Tray, the Lower Wedge Tray, the “Client” Attenuating Material selected and the “Client” Linear Attenuation Coefficient calculated for that “Client” Accelerator ID for X and Y tool location and its depth of cut “Z” value for the attenuating material and Tray location.

Each tooling strategy for the “Finish Cut” removal of material in the “Converter Database” for this “MSB” tooling instructions comprises at least one, ⅛″ Diameter Ball-End-Mill Tool associated therewith to be used by the tooling machine. Each tooling strategy is defined based upon the conversion of a Digital Imaging and Communications in Medicine Radiation Therapy (DICOM RT) Cancer Patient Treatment Plan into multiple Treatment Field 2D Matrices and into multiple IMRT Filter (Compensator) 3 Dimensional Matrices and into 3 Dimensional Computer Numerical Code (CNC) Files and providing a set of Intensity Modulated Radiation Filters (IMRT Filters); IMRT Filters and/or IMRT Radiation Blocking Filters used for each Cancer Patient's Treatment Field angle; and/or Radiation Wound Protective Blocking Filters used for each Cancer Patient's Surgical Incision, to be used by the Radiation Oncology Hospital or Clinic (e.g.; a “Client”) to modulate the radiation dose for the Cancer Patient for a specific radiation accelerator treatment machine.

The strategy for each “Finish Cut” tool is automatically matched within the “Converter Database”; to the “Client” ID, “Client” Address, “Client” Contact Information, “Client” Accelerator ID, selection of an Attenuating material for the “Client”, Linear Attenuation Coefficient (LAC) calculated for that material and specific radiation accelerator treatment machine, the Tray selected for mounting the IMRT Filter, Upper Wedge Tray Distance from Source, Wedge Tray Thickness, Block Tray Distance from Source, Block Tray Thickness, Lower Wedge Tray Distance from Source, Lower Wedge Tray Thickness, Isocenter Distance from Source, Isocenter Calibration Markings, X1 X2 and Y1 Y2, on the Upper Wedge Tray, the Block Tray, the Lower Wedge Tray, the “Client” Attenuating Material selected and the “Client” Linear Attenuation Coefficient calculated for that “Client” Accelerator ID for X and Y tool location and its depth of cut “Z” value for the attenuating material and Tray location.

Each tooling strategy for the “Location Hole Cut” removal of material in the “Converter Database” for this “MSB” tooling instructions comprises at least one Letter F Drill Tool associated therewith to be used by the tooling machine. Each tooling strategy is defined based upon the conversion of a Digital Imaging and Communications in Medicine Radiation Therapy (DICOM RT) Cancer Patient Treatment Plan into multiple Treatment Field 2D Matrices and into multiple IMRT Filter (Compensator) 3 Dimensional Matrices and into 3 Dimensional Computer Numerical Code (CNC) Files and providing a set of Intensity Modulated Radiation Filters (IMRT Filters); IMRT Filters and/or IMRT Radiation Blocking Filters used for each Cancer Patient's Treatment Field angle; and/or Radiation Wound Protective Blocking Filters used for each Cancer Patient's Surgical Incision, to be used by the Radiation Oncology Hospital or Clinic (e.g.; a “Client”) to modulate the radiation dose for the Cancer Patient for a specific radiation accelerator treatment machine.

The strategy for each “Location Hole Cut” tool is automatically matched with the “Converter Database”; for the “Client” ID, “Client” Address, “Client” Contact Information, “Client” Accelerator ID, selection of an Attenuating material for the “Client”, Linear Attenuation Coefficient (LAC) calculated for that material and specific radiation accelerator treatment machine, the Tray selected for mounting the IMRT Filter, Upper Wedge Tray Distance from Source, Wedge Tray Thickness, Block Tray Distance from Source, Block Tray Thickness, Lower Wedge Tray Distance from Source, Lower Wedge Tray Thickness, Isocenter Distance from Source, Isocenter Calibration Markings, X1 X2 and Y1 Y2, on the Upper Wedge Tray, the Block Tray, the Lower Wedge Tray, the “Client” Attenuating Material selected and the “Client” Linear Attenuation Coefficient calculated for that “Client” Accelerator ID for X and Y tool location and its depth of cut “Z” value for the attenuating material and Tray location.

Each tooling strategy for the “Engraving” in the “Converter Database” for this “MSB” tooling instructions comprises at least one Engraving Tool associated therewith to be used by the tooling machine. Each tooling strategy is defined based upon the conversion of a Digital Imaging and Communications in Medicine Radiation Therapy (DICOM RT) Cancer Patient Treatment Plan into multiple Treatment Field 2D Matrices and into multiple IMRT Filter (Compensator) 3 Dimensional Matrices and into 3 Dimensional Computer Numerical Code (CNC) Files and providing a set of Intensity Modulated Radiation Filters (IMRT Filters); IMRT Filters and/or IMRT Radiation Blocking Filters used for each Cancer Patient's Treatment Field angle; and/or Radiation Wound Protective Blocking Filters used for each Cancer Patient's Surgical Incision, to be used by the Radiation Oncology Hospital or Clinic (e.g.; “Client”) to modulate the radiation dose for the Cancer Patient for a specific radiation accelerator treatment machine.

The strategy for each “Engraving” tool is automatically matched with the “Converter Database”; for the “Client” ID, “Client” Address, “Client” Contact Information, “Client” Accelerator ID, selection of an Attenuating material for the “Client”, Linear Attenuation Coefficient (LAC) calculated for that material and specific radiation accelerator treatment machine, the Tray selected for mounting the IMRT Filter, Upper Wedge Tray Distance from Source, Wedge Tray Thickness, Block Tray Distance from Source, Block Tray Thickness, Lower Wedge Tray Distance from Source, Lower Wedge Tray Thickness, Isocenter Distance from Source, Isocenter Calibration Markings, X1 X2 and Y1 Y2, on the Upper Wedge Tray, the Block Tray, the Lower Wedge Tray, the “Client” Attenuating Material selected and the “Client” Linear Attenuation Coefficient calculated for that “Client” Accelerator ID for X and Y tool location and its depth of cut “Z” value for the attenuating material and Tray location.

Each set of “Engraving” tooling instructions taken from the “Converter Database” using an Automated Programming Interface (API) further comprising the engraving of the Isocenter X1 X2 and Y1 Y2 in relationship to the Isocenter on the source side surface of the Intensity Modulated Radiation Filter; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for the specific radiation accelerator treatment machine, for the specific tray location, selected mounting position and specific Linear Attenuation Coefficient, automatically taken from the “Converter Database” for that “Client”, for that Accelerator, for that Tray location, and for that attenuating material and reducing human error.

Each set of “Engraving” tooling instructions taken from the “Converter Database” using an Automated Programming Interface (API) further comprising the engraving the CNC Machine Identification Number assigned on the source side surface X2 Y1 quadrant of the Intensity Modulated Radiation Filter; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient, automatically taken from the “Converter Database” for that “Client”, for that Accelerator, for that Tray location, and for that attenuating material and reducing human error.

Each set of Probe tooling instructions taken from the “Converter Database” using an Automated Programming Interface (API) further comprises a tooling strategy for the “Tolerance Calibration” QA which comprises at least one Inspection Probe Tool associated therewith to be used by the tooling machine. Each Probe tooling strategy is defined based upon the conversion of a Digital Imaging and Communications in Medicine Radiation Therapy (DICOM RT) Cancer Patient Treatment Plan into multiple Treatment Field 2D Matrices, into multiple IMRT Filter (Compensator) 3 Dimensional Matrices, and into 3 Dimensional Computer Numerical Code (CNC) Files. Thereafter there is provided a set of Intensity Modulated Radiation Filters (IMRT Filters); IMRT Filters and/or IMRT Radiation Blocking Filters used for each Cancer Patient's Treatment Field angle; and/or Radiation Wound Protective Blocking Filters used for each Cancer Patient's Surgical Incision, to be used by the Radiation Oncology Hospital or Clinic (e.g.; “Client”) to modulate the radiation dose for the Cancer Patient for a specific radiation accelerator treatment machine.

The strategy for each Probe tool is automatically matched with the Converter Database; “Client” ID, “Client” Address, “Client” Contact Information, “Client” Accelerator ID, selection of an Attenuating material for the “Client”, Linear Attenuation Coefficient (LAC) calculated for that material and specific radiation accelerator treatment machine, the Tray selected for mounting the IMRT Filter, Upper Wedge Tray Distance from Source, Wedge Tray Thickness, Block Tray Distance from Source, Block Tray Thickness, Lower Wedge Tray Distance from Source, Lower Wedge Tray Thickness, Isocenter Distance from Source, Isocenter Calibration Markings, X1 X2 and Y1 Y2, on the Upper Wedge Tray, the Block Tray, the Lower Wedge Tray, the “Client” Attenuating Material selected and the “Client” Linear Attenuation Coefficient calculated for that “Client” Accelerator ID for X and Y tool location and its depth of cut “Z” value for the attenuating material and Tray location.

Each set of Probe tooling instructions taken from the “Converter Database” using an Automated Programming Interface (API). The Z value for nine inspection points is measured on the Intensity Modulated Radiation Filter (IMRT Filter) after machining, as defined within the inspection database based upon the nine measured inspection points and the received converted DICOM RT Cancer Patient Treatment Plan Field CNC (“G-Code”)

Data for each IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic (e.g.; “Client”) for a specific radiation accelerator treatment machine, a specific tray location, and a specific Linear Attenuation Coefficient. Thereafter there is generated a quality assurance report based upon the defined inspection database for that “Client”, for that Accelerator, for that Tray location, and for that attenuating material and reducing human error.

The API also includes a presentation of a quality assurance report that is based upon measuring the acceptable tolerances of the nine inspection points on the Intensity Modulated Radiation Filter (IMRT Filter) after machining, and automatically making a comparison to an inspection database based upon the nine measured inspection points calculated from the received DICOM RT Cancer Patient Treatment Plan Data or the 3 Dimensional Compensator Data received from the “Client”. The quality assurance report is automatically generated based upon the defined inspection database reducing any human error.

The API, when the acceptable tolerances have been met, includes defining that the IMRT Filter Compensator is cleaned and placed at the end of the Shipping Queue. When the quality assurance report is satisfactory, then the Intensity Modulated Radiation Filter (IMRT Filter) is shipped using a “Tracking Number” and Confirmation, via the Internet using a secure “MSB” SFTP Server, is sent both to the “Client” and the Converter confirming that the set of Intensity Modulated Radiation Filters (IMRT Filters) have been shipped.

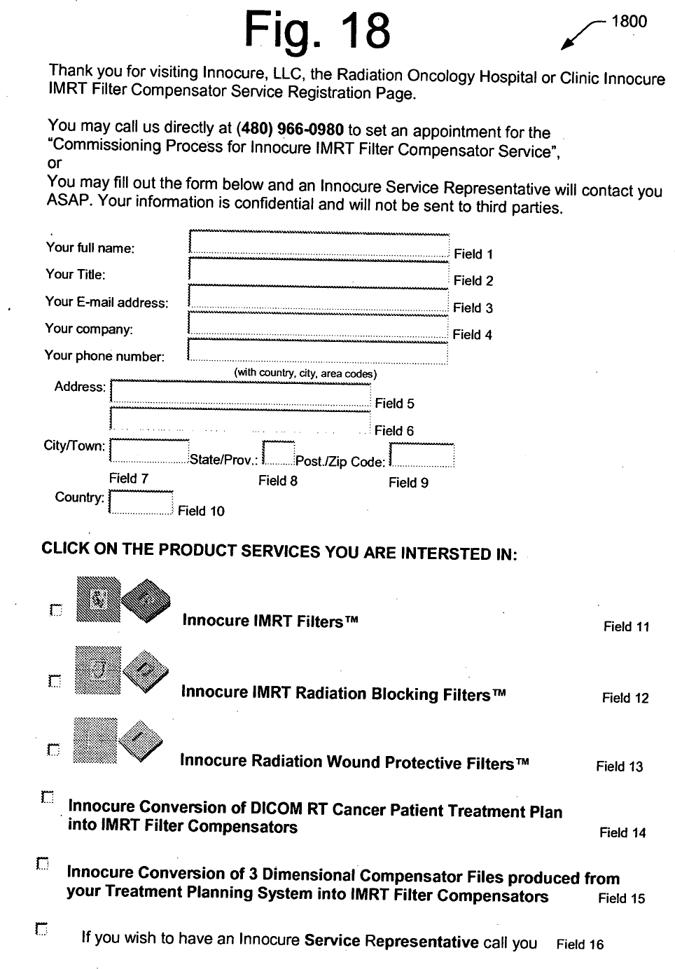

FIG. 18 depicts, in a table of input fields, an exemplary user interface display screen for an IMRT Filter Compensator Service Registration, the foregoing being provided, for instance, as a Converter Web Site Interface for the “Client” and the “MSB”. FIG. 19 depicts, in a table of input fields for shipping/invoice/email address, contact names, and Tel/fax, an exemplary user interface display screen for an IMRT Filter Compensator Service Registration intended for used by radiation oncology units at hospitals or clinics. FIG. 20 depicts, in a table of input fields for a Header File containing Information for a Patient Treatment Plan, an exemplary user interface display screen. Note that, relative to FIG. 20, the information in the given examples of the DICOM RT Codes are taken from the DICOM RT Patient Treatment Plan. Also note that:
(i) An RT Dose IOD referenced within the Referenced Dose Sequence (300C,0080) can be used for storing grid-based (pixel) data, isodose curves, and/or individual dose points (with optional dose point names) for the current Fraction Group; and
(ii) The Meterset at a given Control Point (see RT Beams Module) is equal to the Beam Meterset (300A,0086) multiplied by the Cumulative Meterset Weight (300A,0134) for the Control Point, divided by the Final Cumulative Meterset Weight (300A,010E).

Another illustrated embodiment consisting of display formats on a computer screen for processing and tracking a received secure “Client” SFTP Server request for an Intensity Modulated Radiation Filter (IMRT Filter). The display formats will now be described with reference to FIG. 20. FIG. 20 is labeled as being the DICOM RT IMRT Filter Patient Compensator File Header Information for tracking the received DICOM RT IMRT Filter Files via the Internet using a secure “Client” SFTP Server and a secure “MSB” SFTP Server that is divided into four separate sections referred to below in FIGS. 21a, 21b, and 22. Namely, in these Figures, there is a table for a “Client” Order Tab, a table for a Converter Conversion Tab, a table for a “MSB” Manufacturing Tab, and a table for a Shipping Order/Confirmation Tab. Each of the four sections is represented by a Tab, and selection of any particular Tab causes the corresponding display screen to be displayed.

FIG. 21a depicts, in a table of input fields for a “Client” Order Tab, an exemplary user interface display screen. The “Client” Order Tab, field 60 is selected, and the display field 61 provides a specific job number, which has been incremented from the previous job number, (example: 0011 1211 115 1003 070706 1151 11 00101), assigned to this “Client” for each order along with the “Client” identification number, field 60. The “Client” identification number is automatically correlated using the API to the “Converter Database” of “Clients” for placing the order, including the Cancer Patient's Identification Number including the Patient's Name, Sex and Birth Date for the corresponding Intensity Modulated Radiation Filter (IMRT Filter).

Referring to FIG. 21a, the “Client” Order Tab provides a field 60 indicating that the secure “Client” SFTP Server used to access the “Converter Database” by an Automated Programming Interface (API) was read and accepted, and a job identification number field 61 was created. Field 62 indicates that a directory structure was created, and the DICOM RT Cancer Patient Treatment Plan Data has been copied into that directory. Field 63 indicates that the “Client” placing the order is in a “Client” database, and field 64 indicates that the radiation therapy machine intending to use the Intensity Modulated Radiation Filter (IMRT Filter) is in the “Client” database. If the radiation therapy machine is not in the “Client” database, then an error message is automatically generated to initiate a Converter “Commissioning Process”. Field 65 indicates that a “MSB” xml work order has been processed. Field 66 can be selected if the “MSB” xml work order needs to be reprocessed. Field 67 can be selected if the “MSB” xml work order needs to be cancelled. Approval of the items corresponding to the “Client” Order Tab is provided in field 68.

FIG. 21b depicts, in a table of input fields for a Conversion Tab, an exemplary user interface display screen. The Converter Conversion Tab, field 70, is selected in Table 5 and the display repeats the same information as in field 60. Field 71 indicates if the API has been successfully run. The API reads the “Client's” DICOM RT Cancer Patient Treatment Plan Data. Field 72 indicates that an Intensity Modulated Radiation Filter (IMRT Filter) blank has been selected. Field 73 indicates that the Intensity Modulated Radiation Filter (IMRT Filter) CNC 3 Dimensional Milling Instructions program has been programmed. Field 74 indicates that Nine-Point inspection program file for the Intensity Modulated Radiation Filter (IMRT Filter) has been programmed. Field 75 indicates that the Intensity Modulated Radiation Filter (IMRT Filter) CNC 3 Dimensional Milling Instructions program has been verified. Field 76 indicates that the Intensity Modulated Radiation Filter (IMRT Filter) Nine-Point inspection program file has been verified. Field 77 indicates for which “MSB” the post processed file will be sent to. Approval of the items corresponding to the Converter Engineering Tab is provided in field 78.

FIG. 22 depicts, in a table of input fields for an “MSB” Manufacturing Tab, an exemplary user interface display screen. Once the Converter receives the DICOM RT Cancer Patient Treatment Plan via the secure “Client” SFTP Server, and automatically converts the Treatment Plan into multiple Treatment Plan Fields resulting in a “Converter Database” structure comprising DICOM RT Compensator Thickness Matrices and Compensator Transmission Matrices for return to the “Client for Dose Calculations. The converter automatically converts each Treatment Plan Field into 3 Dimensional matrix Compensator Files. The converter automatically converts each 3 Dimensional matrix Compensator File into CNC “G-Code” Milling Instruction Files. The converter automatically converts each CNC “G-Code” Milling Instruction Files into Nine-Point Probe Testing files. In addition the converter automatically converts the IMRT Files into a set of computer-readable Barcode Labels. Each Barcode Label contains the information to automatically retrieve the “Milling and Testing” files via the secure “MSB” SFTP Server to manufacture each Intensity Modulated Radiation Filter (IMRT Filter) for the exact “Client”, the exact radiation accelerator treatment machine, the exact tray location, the exact linear attenuation coefficient, the exact Patient, and the correct Treatment Field. The information required for the Barcode Label is automatically converted directly from the DICOM RT Cancer Patient's Treatment Plan (Description* see the DICOM Tags below), stored in the Converter Database.

The “MSB” Manufacturing Tab, field 80, is selected in FIG. 22 (Table 6), and the display repeats the same information as in fields 60 and 70. Field 81 indicates if the Converter “MSB” Purchase Order number has been assigned. Field 82 indicates the set of IMRT Filter Labels have been printed. Field 83 indicates the IMRT Filter labels have been attached to the correct blanks. Field 84 indicates that the Blank IMRT Filters with labels have been placed in the “MSB” FIFO Production Queue. Field 85 indicates that an IMRT Filter is being machined. Field 86 indicates that an inspection test has been achieved. Field 87 indicates that the Intensity Modulated Radiation Filter (IMRT Filter) has been removed, checked, cleaned and placed at the end of the “MSB” Shipping Queue. Approval of the items corresponding to the “MSB” Manufacturing Tab is provided in field 88.

In order for the “MSB” to manufacture an IMRT Filter, the “MSB” uses a Barcode Reader to scan the next IMRT Filter Label in the “MSB” Production FIFO Queue, using the API via the Internet using a secure “MSB” SFTP Server. This results in the automatic retrieval from the “Converter Database” the following structures (highlighted in red) comprising the CNC tooling instructions to automatically mill the IMRT Filter, for the exact “Client”, for the exact Patient, for the exact radiation accelerator treatment machine, for the exact tray location, correct Tray mounting position, for the exact attenuating material, and for the exact Treatment Field.

Use the Bar Code for the Order ID

| Aaab | cdef | ghi | xxxz | ddmmyy | lmno | pq | jjjkk |
|---|---|---|---|---|---|---|---|
| 0011 | 1211 | 115 | 1003 | 070706 | 1151 | 11 | 00101 |

aaa “Client”

b “Cient” Accelerator Name ID

g Filter Type
h IMRT Material ID

i Number of Filters

xxxz Patient ID#

yy Year

n = Number of Beams (Number of
Filters)

q Beam Number

(Priority will not be allowed in the first 4 Months we will use First In First Out—FIFO)

<address>

<location>Virginia G. Piper Cancer Center</location>
<address1>10460 N 92nd Street</address1>
<address2>Attn: Dr. Smith</address2>
<city>Scottsdale</city>
<state>AZ</state>
<country>USA</country>
<postal>85258</postal>

</address>

This will come from information in the Barcode (See Fig. 1a)

<filters count=“3” type=“block”> (This TYPE information will come
from See Figs. 1a and 18)

<filter id=“001” barcode=“14320577621001”/> (Beam Number)
<filter id=“002” barcode=“14320577621002”/> (Beam Number)
<filter id=“003” barcode=“14320577621003”/> (Beam Number)

</filters>
<notes>This is a test order</notes>
</order>.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Possible Claims 1 to 35 that could be set forth in this application are listed below on the following pages 59 to 74 and are incorporated herein as part of the disclosure. The only Claim currently being set forth and pursued, however, is Claim 1 on page 74a.

1. A method for building a secure “Client” SFTP Server for access to the “Converter Database” and for building a secure “MSB” SFTP Server for access to the “Converter Database” to provide a secure “Client” Cancer Patient IMRT Filter Compensator Service, via the Internet using an Automated Programming Interface (API).

2. A method according to claim 1 for building a secure “Client” SFTP Server for access to the “Converter Database” by a “Commissioning Process”, which includes; establishing an approved Radiation Oncology Hospital or Clinic (hereinafter referred to as “Client”); “Client” ID, “Client” Address, “Client” Contact Information, “Client” Accelerator ID, selection of an attenuating material for the “Client” (Tungsten Metal Powder, Cerrobend, Aluminum 6061T6 or Brass 360), Linear Attenuation Coefficient (LAC) calculated for that attenuating material and specific radiation accelerator treatment machine, the Tray selected for mounting the IMRT-Filter (Upper Wedge Tray Distance from Source, Wedge Tray Thickness, Block Tray Distance from Source, Block Tray Thickness, Lower Wedge Tray Distance from Source, Lower Wedge Tray Thickness), the mounting position on the selected Tray (source side or patient side), the Isocenter Distance from Source, Isocenter Calibration Markings (X1 X2 and Y1 Y2), on the selected Tray, and assigning a “Client” Password in the “Converter Database” for access to the secure “Client” SFTP Server.

3. A method according to claim 1 and claim 2 for providing a set of Intensity Modulated Radiation Filters (IMRT Filters); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, mounting position and specific Linear Attenuation Coefficient, which includes; receiving via the Internet using a secure “Client” SFTP Server the DICOM RT Cancer Patient Treatment Plan, including the optimal fluence field data for the multiple Treatment Fields (angles) and converting the DICOM RT Cancer Patient Treatment Plan into a DICOM RT Cancer Patient Treatment Plan with IMRT Filters (Compensators—with Thickness Matrices and Transmission Matrices in the DICOM format); or, receiving via the Internet using a secure “Client” SFTP Server the 3 Dimensional Compensator Files for conversion; wherein the steps comprise:
(i) Receipt (IMPORT), from an approved Radiation Oncology Hospital or Clinic (e.g.; “Client”) of a DICOM RT Cancer Patient Treatment Plan File via the Internet using a secure “Client” SFTP Server.
(ii) Conversion of the DICOM RT Cancer Patient Treatment Plan File into a DICOM RT-Cancer Patient Treatment Plan IMRT Filter Compensator File; Compensators—with Thickness Matrices and Transmission Matrices in the DICOM format for returning (EXPORT) to the Radiation Oncology Hospital or Clinic (“Client”) for Dose Calculation via the Internet using a secure “Client” SFTP Server.
(iii) Conversion of the DICOM RT Cancer Patient Treatment Plan Optimal Fluence Field Files into Cancer Patient Treatment Plan 3 Dimensional CNC Compensator Milling (“G-Code”) Files for sending (EXPORT), via the Internet using a secure “MSB” SFTP Server, to the Manufacturing Service Bureau (MSB) for milling the IMRT Filter Compensators for the Client.
(iv) Receipt (IMPORT) from an approved Radiation Oncology Hospital or Clinic “Client” multiple Cancer Patient Treatment 3 Dimensional Compensator Files for a Cancer Patient, via the Internet using a secure “Client” SFTP Server.
(v) Conversion of the multiple Cancer Patient Treatment 3 Dimensional Compensator Files into 3 Dimensional CNC Compensator Milling (“G-Code”) Files for sending (EXPORT) via the Internet using a secure “MSB” SFTP Server to the Manufacturing Service Bureau (MSB) for milling the IMRT Filter Compensators for the Client.

4. A method according to claim 3 wherein each DICOM RT Cancer Patient Treatment Plan File is converted into a DICOM RT Cancer Patient Treatment Plan IMRT Filter Compensator File (Compensators—with Thickness Matrices and Transmission Matrices in the DICOM format) and stored into the “Converter Database” and exported back to the “Client”, via the Internet using a secure “Client” SFTP Server, for Dose Calculations.

5. A method according to claim 3 wherein each Intensity Modulated Radiation Filter, IMRT Filter and/or IMRT Radiation Blocking Filter used for each Cancer Patient's Treatment Field angle; and/or Radiation Wound Protective Blocking Filter used for each Cancer Patient's Surgical Incision, for a specific accelerator, for a specific Radiation Oncology Hospital or Clinic “Client”, is converted from the DICOM RT Cancer Patient Treatment Plan Optimal Fluence Field Files into Cancer Patient Treatment Plan 3 Dimensional CNC Compensator Milling (“G-Code”) Files and stored within the “Converter Database” and exported, via the Internet using a secure “MSB” SFTP Server, to the Manufacturing Service Bureau (MSB) for milling the IMRT Filter Compensators for the “Client”.

6. A method according to claim 3 wherein each set of multiple Cancer Patient Treatment 3 Dimensional Compensator Files for a Cancer Patient are received, via the Internet using a secure “Client” SFTP Server, and converted into Cancer Patient Treatment Plan 3 Dimensional CNC Compensator Milling (“G-Code”) Files and stored into the “Converter Database” and exported, via the Internet using a secure “MSB” SFTP Server, to the Manufacturing Service Bureau (MSB) for milling the IMRT Filter Compensators for the “Client”.

7. A method according to claim 3 wherein the Intensity Modulated Radiation Filter, IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, Linear Attenuation Coefficient (LAC) requirement corresponds to the exact “Client” and exact radiation accelerator treatment machine stored in the “Converter Database”.

8. A method according to claim 3 wherein the Intensity Modulated Radiation Filter, IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, Tray requirements corresponds to the exact “Client” and exact radiation accelerator treatment machine stored in the “Converter Database”.

9. A method according to claim 3 wherein the Intensity Modulated Radiation Filter, IMRT Filter and/or IMRT Radiation Blocking Filter used for each Cancer Patient's Treatment Field angle; and/or Radiation Wound Protective Blocking Filter used for each Cancer Patient's Surgical Incision, for a specific accelerator, for a specific Radiation Oncology Hospital or Clinic “Client”, Database comprises a “Client” ID/“Client” Address/“Client” Accelerator Name/“Client” Accelerator Serial Number/Beam Number/Attenuating material/Linear Attenuating Coefficient (LAC)/Manufacturing Service Bureau (MSB) Name/Patient Name/Patient Birth Date/Patient Sex/Patient Study Date/Number of Beams/Treatment Field Number/Planned Target Volume (hereinafter referred to as “IMRT Filter Number”).

10. A method according to claim 9 resulting in an “Converter Database” structure comprising: a computer-readable Barcode medium described below; wherein, each Intensity Modulated Radiation Filter (IMRT Filter) corresponds to at least one particular type radiation accelerator treatment machine, each type radiation accelerator treatment machine having an IMRT Filter size requirement dependent on the attenuating material selected, the Tray location selected, and the calculated linear attenuation coefficient previously stored; wherein the bar code comprises:

| Name | IMRT Filter Label Barcode (Value) | Dicom Tag |
|---|---|---|
| “Client” ID* | aaab cdef ghi xxxz ddmmyy lmno pq jjjkk | |
| | InstitutionName | (0008, 0080) |
| Accelerator Name ID* | aaab cdef ghi xxxz ddmmyy lmno pq jjjkk | |
| | TreatmentMachineName | (300A, 00B2) |
| Accelerator SN ID* | aaab cdef ghi xxxz ddmmyy lmno pq jjjkk | |
| | DeviceSerialNumbcr | (0018, 1000) |
| Tray Selection* | aaab cdef ghi xxxz ddmmyy lmno pq jjjkk | |
| | d = 1 = Upper Wedge Tray* | (300A, 00DA) |
| | d = 2 = Block Tray* | (300A, 00F6) |
| | d = 3 = Lower Wedge Tray* | (300A, 00E6) |
| Tray Distance* | aaab cdef ghi xxxz ddmmyy lmno pq jjjkk | |
| | SourceToCompensatorTrayDistance: | (300A, 00E6) |
| Mounting IMRT Filter* | aaab cdef ghi xxxz ddmmyy lmno pq jjjkk | |
| | CompensatorMountingPosition* | (300A, 02E1) |
| | f = 1 = Radiation Source Side* | |
| | f = 2 = PatientSide* | |
| Filter Type* | aaab cdef ghi xxxz ddmmyy lmno pq jjjkk | (300A, 00F8) |
| | g = 1 = IMRT Filter | |
| | g = 2 = IMRT Radiation Blocking Filter | |
| | g = 3 = Radiation Wound Protective Filter | |
| IMRT Material ID* | aaab cdef ghi xxxz ddmmyy lmno pq jjjkk | |
| | MaterialID* | (300A, 00E1) |
| | h = 1 = Tungsten Powdered Metal | |
| | h = 2 = Molten Cerrobend | |
| | h = 3 = Aluminum (6061T6) | |
| | h = 4 = Brass 360 | |
| | h = 5 = Custom | |
| Number of Filters* | aaab cdef ghi xxxz ddmmyy lmno pq jjjkk | |
| | NumberOfCompensators* | (300A, 00E0) |
| | i = 0 = 10 IMRT Filters | |
| | i Max = 10 | |
| | i Min = 3 | |
| Patient ID#* | aaab cdef ghi xxxz ddmmyy lmno pq jjjkk | (0010, 0020) |
| | z = 1 = PatientName* | (0010, 0010) |
| | z = 2 = PatientSex* | (0010, 0040) |
| | z = 3 = PatientBirthDate* | (0010, 0030) |
| Patient Plan Date* | aaab cdef ghi xxxz ddmmyy lmno pq jjjkk | |
| | StudyDate* | (0008, 0020) |
| | StudyTime* | (0008, 0030) |
| Fluence Plan Number* | aaab cdef ghi xxxz ddmmyy lmno pq jjjkk | |
| | l = ReferencedBeamNumber* | (300C, 0006) |
| | m = BeamMeterset* | (300A, 0086) |
| | n = NumberOfBeams* | (300A, 0080) |
| | o = BeamSequence* | (300A, 00B0) |
| Fluence Plan Field Number* | aaab cdef ghi xxxz ddmmyy lmno pq jjjkk | |
| | p = BeamName* | (300A, 00C2) |
| | q = BeamNumber* | (300A, 00C0) |
| “MSB” ID** | aaab cdef ghi xxxz ddmmyy lmno pq jjjkk | |
| “MSB” Quantity of CNC** | aaab cdef ghi xxxz ddmmyy lmno pq jjjkk | |
| | kk = Number of CNC Machines | |

*Taken automatically from DICOM RT Patient Treatment Plan and Database
**Taken automatically from Database

11. A method according to claim 5 wherein defining the CNC “G-Code” Tooling Instructions comprises the conversion of the DICOM RT Cancer Patient Treatment Field Optimal Fluence Plan Data 2D Matrices into multiple 3 Dimensional Matrices by using the IMRT Filter Type, Attenuating Material selected, Linear Attenuation Coefficient (LAC), Tray location, Filter mounting position, Maximum Depth of Cut (MDC), Patient Isocenter Distance (PID), stored in the “Converter Database” for this “Client” and for this radiation accelerator treatment machine.

12. A method according to claim 7, claim 8 and claim 9 wherein defining the multiple 3 Dimensional Matrices by translating the Cancer Patient Isocentric 2D Matrix XY values to the Tray locations for the IMRT Filter, i.e., Upper Wedge Tray Distance from Source (UWTD), Block Tray Distance from Source (BTD), and Lower Wedge Tray Distance from Source (LWTD), stored in the “Converter Database” for this “Client” and for this radiation accelerator treatment machine.

13. A method according to claim 11 wherein defining these new Matrix X and Y values are created by the following formulas UWTD/PID times the old X and Y Values, BTD/PID times the old X and Y Values, and LWTD/PID times the old X and Y Values.

14. A method according to claim 11 wherein defining the multiple 3 Dimensional Matrices by translating the normalized Z value calculated by the “Client” Treatment Planning System in each 2D matrix cell by translating the Cancer Patient Isocentric 2D Normalized Z values into the Tray locations for the IMRT Filter, i.e., Upper Wedge Tray Distance from Source (UWTD), Block Tray Distance from Source (BTD), and Lower Wedge Tray Distance from Source (LWTD). These new Z values are created by the following formula:
New Z=Maximum Depth of Cut+PID*log((Old Z)/LAC/(sqrt((new X*new X)+(new Y*new Y)+1000000.0))).

15. A method according to claim 13 and claim 14 wherein defining the new multiple 3 Dimensional Matrices by translating the XYZ values into CNC “G-Code” tooling instructions for milling the IMRT Filters.

16. A method according to claim 1 for building a secure “MSB” SFTP Server for access to the “Converter Database” by a “MSB Evaluation Process”, which includes; establishing a “MSB” ID, “MSB” Address, “MSB” contact information, “MSB” Password, “MSB” CNC Machine IDs, the Number of CNC Machines, the “Rough Cut” Tool, the “Finish Cut” Tool, the “Location Hole” Tool, the Engraving Tool, and the “Inspection Probe” Tool.

17. A method according to claim 16 for providing a set of Intensity Modulated Radiation Filters (IMRT Filters); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position, and specific Linear Attenuation Coefficient, which includes; sending via the Internet using a secure “MSB” SFTP Server the CNC “G-Code” tooling instructions for milling the IMRT Filters, wherein the removal of material to form the IMRT Filter in the tooling database comprises five separate machine tools to apply five separate milling tasks in the creation of an IMRT Filter and providing 6-sigma quality control to meet acceptable tolerances.

18. A method according to claim 15, claim 16 and claim 17 wherein, when the next “MSB” CNC Machine becomes available, the “MSB” Technician selects the next IMRT Filter Blank for the “MSB” Production FIFO Queue and places it into the CNC Machine Fixture. The Technician then uses a Barcode Reader to scan the IMRT Filter Label to automatically retrieve the CNC Milling Code from the “Converter Database”, via the secure “MSB” SFTP Server, to mill the IMRT Filter Compensator for a specific “Client”, for a specific accelerator, for a specific Attenuating Material, for a specific Tray Location, and for this Patient's specific Treatment Angle. This method eliminates any possibility of Human Error.

19. A method according to claim 18 wherein first, the “Rough Cut” tool instructions automatically retrieved for removal of material to form the IMRT Filter in the tooling database comprises at least one tool, a ¼″ ball-end-mill, associated therewith to be used by the automated CNC machine. This automatic download method eliminates any possibility of Human Error.

20. A method according to claim 18 wherein second, the “Finish Cut” tool instructions automatically retrieved for removal of material to form the IMRT Filter in the tooling database comprises at least one tool, a ⅛″ ball-end-mill, associated therewith to be used by the automated CNC machine. This automatic download method eliminates any possibility of Human Error.

21. A method according to claim 18 wherein third, the “Location Hole” tool instructions automatically retrieved for removal of material to form the IMRT Filter in the tooling database comprises at least one tool, a Letter F Drill, associated therewith to be used by the automated CNC machine. This automatic download method eliminates any possibility of Human Error.

22. A method according to claim 18 wherein fourth, the “Engraving” tool instructions for automatically retrieved removal of material to form the IMRT Filter in the tooling database comprises at least one tool, an Engraving Tool, associated therewith to be used by the automated CNC machine. This automatic download method eliminates any possibility of Human Error.

23. A method according to claim 18 wherein fifth, the “Tolerance Calibration” tool instructions automatically retrieved for to test the depth of cut at selected XY locations within the IMRT Filter in the tooling database comprises at least one tool, an “Inspection Probe”, associated therewith to be used by the automated CNC machine. This automatic download method eliminates any possibility of Human Error.

24. A method according to claims 17 to 23 wherein each set of tooling instructions are defined based upon the 3 Dimensional XYZ data converted from the specific “Client” DICOM RT Cancer Patient Treatment Plan for the “Client” specific accelerator, “Client” tray location, selected mounting position and size of the Intensity Modulated Radiation Filter (IMRT Filter) blank and the “Client” attenuating material selected for the blank. This method includes the conversion of the optimal fluence field data for each Cancer Patient Treatment Field angle into a 3 Dimensional Computer Numerical Code (CNC) Machine Control Files to be used to control the milling of the Intensity Modulated Radiation Filter (IMRT Filters, IMRT Radiation Blocking Filters and Radiation Wound Protective Blocking Filters) used for each Cancer Patient's treatment angle. This CNC Machine Control File is sent (exported) via the Internet using a secure “MSB” SFTP Server to the Manufacturing Service Bureau (MSB) for manufacturing the Cancer Patient's Set of Intensity Modulated Radiation Filters (IMRT Filters); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient.

25. A method according to claims 17 to 23 wherein each set of tooling instructions are defined based upon the 3 Dimensional XYZ data converted from the 3 Dimensional Compensator Files received (imported) from “Clients” that have Treatment Planning Systems that can produce Compensator Files via the Internet using a secure “Client” SFTP Server and convert them into 3 Dimensional Computer Numerical Code (CNC) Machine Control Files to be used to control the milling of the Intensity Modulated Radiation Filter (IMRT Filters, IMRT Radiation Blocking Filters and Radiation Wound Protective Blocking Filters) used for each Cancer Patient's treatment angle. This CNC Machine Control File is sent (exported) via the Internet using a secure “MSB” SFTP Server to the Manufacturing Service Bureau (MSB) for manufacturing the Cancer Patient's Set of Intensity Modulated Radiation Filters (IMRT Filters); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient.

26. A method according to claim 21 wherein each set of tooling instructions automatically retrieved further comprise the XY specific hole locations in relationship to the Isocenter for the specific radiation accelerator for the four holds to be drilled through the IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient taken from the “Converter Database” for that “Client”.

27. A method according to claim 22 wherein each set of tooling instructions automatically retrieved further comprise engraving the Isocenter X1 X2 and Y1 Y2 in relationship to the Isocenter for the specific radiation accelerator taken from the “Converter Database” on the source side surface of the Intensity Modulated Radiation Filter; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient.

28. A method according to claim 23 wherein each set of tooling instructions automatically retrieved further comprise engraving the CNC Machine Identification Number assigned, based on the “Converter Database” on the source side surface X2 Y1 quadrant of the Intensity Modulated Radiation Filter; IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, a specific tray location, and a specific Linear Attenuation Coefficient.

29. A method according to claim 23 wherein the measuring of the Z value for Nine-Point Probe inspection points on the Intensity Modulated Radiation Filter (IMRT Filter) after machining; defined within the inspection database based upon the Nine-Point Probe measured inspection points and the received converted DICOM RT Cancer Patient Treatment Plan Field CNC (“G-Code”) Data for each IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient and generating a quality assurance report based upon the defined inspection database for that “Client”, for that Accelerator, for that Tray location, and for that attenuating material.

30. A method according to claim 29 wherein the acceptable tolerances have been met, the IMRT Filter Compensator is cleaned and placed at the end of the Shipping Queue.

31. A method according to claim 30 wherein the “MSB” Shipping Clerk uses the Barcode Reader and packages the set of IMRT Filter Compensators with the matching labels attached to the IMRT Filters and the Shipping Carton/s Label for that Cancer Patient and for that “Client”.

32. A method according to claim 1 further comprising: shipping the set of Intensity Modulated Radiation Filters (IMRT Filters); IMRT Filter and/or IMRT Radiation Blocking Filter used for each Cancer Patient's Treatment Field angle; and/or Radiation Wound Protective Blocking Filter used for each Cancer Patient's Surgical Incision, for a specific Radiation Oncology Hospital or Clinic “Client”, for a specific radiation accelerator treatment machine, specific tray location, selected mounting position and specific Linear Attenuation Coefficient; and sending Shipping/Confirmation and Tracking Number both to the “client” and to via the Internet using a secure “Client” SFTP Server and secure “MSB” SFTP Server that the set of Intensity Modulated Radiation Filters (IMRT Filters) has been shipped.

33. A method according to claim 1 and claim 2 resulting in an “Converter Database” database Automatic Programming Interface (API) structures comprising:
a first data field containing data for receiving a Radiation Oncology Hospital or Clinic, “Client” ID, InstitutionName, via the Internet using a secure “Client” SFTP Server, providing DICOM RT Cancer Patient Treatment Plan Data for a specific radiation accelerator treatment machine;
a second data field containing data for receiving a “Client” Accelerator Name ID, TreatmentMachineName, via the Internet using a secure “Client” SFTP Server, providing DICOM RT Cancer Patient Treatment Plan Data for a specific radiation accelerator treatment machine;
a third data field containing data for receiving a “Client” Accelerator SN ID, DeviceSerialNumber, via the Internet using a secure “Client” SFTP Server, providing DICOM RT Cancer Patient Treatment Plan Data for a specific radiation accelerator treatment machine;
a fourth data field containing data for receiving a “Client” Tray Selection via the Internet using a secure “Client” SFTP Server, providing DICOM RT Cancer Patient Treatment Plan Data for a specific radiation accelerator treatment machine;
a fifth data field containing data for receiving a “Client” Tray Distance, SourceToCompensatorTrayDistance, via the Internet using a secure “Client” SFTP Server, providing DICOM RT Cancer Patient Treatment Plan Data for a specific radiation accelerator treatment machine;
a sixth data field containing data for receiving a “Client” Mounting IMRT Filter, CompensatorMountingPosition, via the Internet using a secure “Client” SFTP Server, providing DICOM RT Cancer Patient Treatment Plan Data for a specific radiation accelerator treatment machine;
a seventh data field containing data for receiving a “Client” Filter Type, IMRT Filter or IMRT Radiation Blocking Filter or Radiation Wound Protective Filter, via the Internet using a secure “Client” SFTP Server, providing DICOM RT Cancer Patient Treatment Plan Data for a specific radiation accelerator treatment machine;
a eighth data field containing data for receiving a “Client” IMRT Material ID, MaterialID, via the Internet using a secure “Client” SFTP Server, providing DICOM RT Cancer Patient Treatment Plan Data for a specific radiation accelerator treatment machine;
a ninth data field containing data for receiving a “Client” Number of Filters, NumberOfCompensators, via the Internet using a secure “Client” SFTP Server, providing DICOM RT Cancer Patient Treatment Plan Data for a specific radiation accelerator treatment machine;
a tenth data field containing data for receiving a “Client” Patient ID#, PatientName, PatientSex and PatientBirthDate, via the Internet using a secure “Client” SFTP Server, providing DICOM RT Cancer Patient Treatment Plan Data for a specific radiation accelerator treatment machine;
a eleventh data field containing data for receiving a “Client” Patient Plan Date, StudyDate and StudyTime, via the Internet using a secure “Client” SFTP Server, providing DICOM RT Cancer Patient Treatment Plan Data for a specific radiation accelerator treatment machine;
a twelfth data field containing data for receiving a “Client” Fluence Plan Number, ReferencedBeamNumber, BeamMeterset, NumberOfBeams and BeamSequence, via the Internet using a secure “Client” SFTP Server, providing DICOM RT Cancer Patient Treatment Plan Data for a specific radiation accelerator treatment machine;
a thirteenth data field containing data for receiving a “Client” Fluence Plan Field Number, BeamName and BeamNumber, via the Internet using a secure “Client” SFTP Server, providing DICOM RT Cancer Patient Treatment Plan Data for a specific radiation accelerator treatment machine;
a fourteenth data field containing data for receiving a Manufacturing Service Bureau, “MSB” ID, via the Internet using a secure “MSB” SFTP Server, providing DICOM RT Cancer Patient Treatment Plan Data for a specific radiation accelerator treatment machine; and
a fifteenth data field containing data for receiving a “MSB” Quantity of CNC Machines, via the Internet using a secure “Client” SFTP Server, providing DICOM RT Cancer Patient Treatment Plan Data for a specific radiation accelerator treatment machine.

34. A method according to claim 1 and claim 2 resulting in a “Converter Database” structure comprising a Purchase Order File called an “order.xml” file—This would be an XML file generated by the Converter. It would contain the Order/Invoice identifier, shipping information (i.e. the hospital address), order priority, the type of filters, the number of filters, the barcode identifier for each filter, and any special processing instructions; wherein an example file is:
<?xml version=″1.0″ encoding=″UTF-8″?>

| <order id=″20060324001″ | | | | | priority=″med″> | | | |
|---|---|---|---|---|---|---|---|---|
| Use the Bar Code for the Order ID | | | | | | | | |
| | aaab | cdef | ghi | xxxz | ddmmyy | lmno | pq | jjjkk |
| | 0011 | 1211 | 115 | 1003 | 070706 | 1151 | 11 | 00101 |

aaa “Client”
b “Cient” Accelerator Name ID
g Filter Type
h IMRT Material ID
i Number of Filters
xxxz Patient ID#
yy Year
n = Number of Beams (Number of
Filters)
q Beam Number
(Priority will not be allowed in the first 4 Months we will use First In First Out—FIFO)
<address>
<location> Virginia G. Piper Cancer Center</location>
<address1>10460 N 92nd Street</address1>
<address2>Attn: Dr. Smith</address2>
<city>Scottsdale</city>
<state>AZ</state>
<country>USA</country>
<postal>85258</postal>
</address>
This will come from the Barcode (See Fig. 1a)
<filters count=“3” type=“block”> (This TYPE information will come
from the Barcode (See Fig. 1a;
<filter id=“001” barcode=“14320577621001”/> (Beam Number)
<filter id=“002” barcode=“14320577621002”/> (Beam Number)
<filter id=“003” barcode=“14320577621003”/> (Beam Number)
</filters>
<notes>This is a test order</notes>
</order>

35. I claim each method, apparatus, device, system, and combinations thereof as illustrated, shown, implied, and described.
